(19) United States
(12) Patent Application Publication
(10) Pub. No.: US 2026/0355532 A1
(43) Pub. Date: Nov. 12, 2026

(54) TREATMENT SYSTEM FOR EYE DISEASE TREATMENT, EYE DISEASE TREATMENT METHOD, AND LIGHT MODULE USED THEREIN

(71) Applicant: MEDI-IOT CO.,LTD., Seoul (KR)

(72) Inventors: Sang Keun LEE, Seoul (KR); Seung Bin PAICK, Hwaseong-si, Gyeonggi-do (KR); Ji Yeol KIM, Seoul (KR)

(73) Assignee: MEDI-IOT CO.,LTD., Seoul (KR)

(21) Appl. No.: 19/476,995

(22) PCT Filed: Jun. 12, 2024
(86) PCT No.: PCT/KR2024/008104

§ 371 Date: Oct. 20, 2025

(30) Foreign Application Priority Data
Jun. 12, 2023 (KR) 10-2023-0074880
Dec. 29, 2023 (KR) 10-2023-0197210
Dec. 29, 2023 (KR) 10-2023-0197211

(51) Int. Cl. A61N 5/06

(52) U.S. Cl. CPC A61N 5/0613; A61N 2005/063; A61N 2005/0648; A61N 2005/0651; A61N 2005/0659; A61N 2005/0661; A61N 2005/0663

(57) ABSTRACT

The present invention relates to eye disease treatment and, more specifically, to a treatment system for eye disease treatment, an eye disease treatment method, and a light module used therein, which treat eye disease by irradiating the eyes with light. The disclosed eye disease treatment system comprises: a main body module (10) in which a light module (100) for generating care light is provided, and which covers the eyes of a user so that the eyes of the user are irradiated with the light of the light module (100); and a wearing module (20) which is coupled to the main body module (10), and which extends to a side surface of the head of the user so as to be worn by the user.

### TECHNICAL FIELD

The present invention relates to eye disease treatment, and more particularly, to a treatment system for eye disease treatment, an eye disease treatment method, and a light module used therein, which treat eye disease by irradiating the eyes with light.

### BACKGROUND ART

As human life expectancy has increased, senile eye diseases are also rapidly increasing.

Representative senile eye diseases, in addition to presbyopia and cataracts that occur with age, include dry eye syndrome, macular edema, macular degeneration, floaters, glaucoma, and nasolacrimal duct obstruction. Recently, the incidence of dry eye syndrome and macular degeneration, which were rare in the past, has been on the rise.

Treatment methods for these eye diseases include intraocular injections, surgery, and eye drops. In the case of procedures or surgeries, there are problems such as surgical side effects like endophthalmitis and vitreous hemorrhage, and symptoms like eye pain, which can make patients feel reluctant about treatment.

To solve these problems, eye treatment devices using light, as disclosed in Patent Documents 1 and 2, have been proposed.

For reference, light with a wavelength of 435-485 nm is known to have a therapeutic effect on mild to moderate acne vulgaris. Furthermore, light with a wavelength of 435-485 nm is known to reduce skin inflammation and activate endogenous porphyrins to generate singlet oxygen, which destroys sebaceous glands and kills P. acnes, the causative agent of acne. In addition, light with a wavelength of 435-485 nm is known to help suppress allergic diseases.

Light with a wavelength of 500-560 nm can be used to treat Bowen's disease and is known to help improve sleep, provide psychological effects, and relieve pain when irradiated on the eyes. Furthermore, light with a wavelength of 500-560 nm is known to be effective in preventing glaucoma, a disease that causes blindness. Glaucoma increases the patient's intraocular pressure, leading to damage to the optic nerve, and green light is effective in lowering this intraocular pressure. In addition, light with a wavelength of 500-560 nm is known to affect nerves and relieve pain from neurological disorders such as migraines.

Furthermore, light with a wavelength of 590 nm is known to inhibit the production of VEGF, i.e. vascular endothelial growth factor. (See the paper “590 nm LED Irradiation Improved Erythema through Inhibiting Angiogenesis of Human Microvascular Endothelial Cells and Ameliorated Pigmentation in Melasma”, Department of Dermatology, Huashan Hospital, Fudan University, 12 Middle Wulumuqi Road, Shanghai 200040, China)

Light with a wavelength of 610-760 nm, particularly 680 nm, is known to relieve inflammation and stimulate skin cells to help produce collagen, an important part of skin recovery. Furthermore, light with a wavelength of 610-760 nm, particularly 680 nm, is known to affect the mitochondria of skin cells, helping to generate adenosine triphosphate, the cell's energy source. Consequently, light with a wavelength of 610-760 nm, particularly 680 nm, generates components that give elasticity to the skin, thereby improving wrinkles, and helps with energy production, improving the skin complexion. It can also be used for improving wound healing, promoting hair growth, and treating psoriasis.

Light with wavelengths of UVA (320-400 nm), UVB (290-320 nm), and UVC (200-0 nm) can be used for psoriasis, vitiligo, atopic dermatitis, and various other inflammatory diseases.

Infrared light has several benefits, including pain relief and tissue repair, and is particularly useful for diabetic patients, being effective in light therapy for wounds due to impaired healing. Furthermore, infrared light is also known to be usable for acne treatment.

Green light can be used to improve sleep, eliminate neuralgia, and treat glaucoma. Additionally, it is known to reduce aggressive behavior in patients with mental illnesses such as dementia and to have an effect on improving depression.

Furthermore, by stimulating cranial nerves through the eyes, it can help prevent neurodegenerative brain diseases such as Parkinson's disease and Alzheimer's disease.

Red light and blue light are known to be able to suppress allergic diseases. It is also effective for allergic rhinitis, a disease that occurs inside the nose. Phototherapy can suppress the effector stage of allergic reactions, and intranasal phototherapy has been proven in clinical trials.

Red light is known to be able to remove bacteria in the oral cavity that cause periodontitis and to promote the regeneration of gums, teeth, and bone after tooth extraction.

By irradiating the inside of the neck with blue light and red light, allergic asthma can be suppressed.

By using red light and blue light, it is known that bacteria inside the ear can be removed to treat related diseases such as otitis externa, and it is also possible to see the effect of treating wounds inside the ear.

In addition, there is research showing that irradiating the inside of the ear with light can stimulate cranial nerves to prevent depression due to seasonal affective disorder.

Infrared light can help in the treatment of hemorrhoids, an anal disease. When infrared light is irradiated on the affected area, it obstructs the blood flow to the hemorrhoids, shrinking the swollen veins, and with consistent treatment, the hemorrhoids can disappear over time.

Irradiation with red light and infrared light is known to be effective in preventing genital diseases and aging in postmenopausal women.

Postmenopausal women experience various problems due to estrogen decline, becoming sensitive to pain and vulnerable to additional diseases such as urinary incontinence due to muscle loss.

Red light can stimulate collagen synthesis in the vaginal tissue and urethral submucosa and promote vasodilation, thereby preventing urinary incontinence and genital-related aging phenomena.

While there are no clear research results for male genitalia, it is known to be able to treat fungal dermatitis that mainly occurs on the skin around the genitals.

Irradiation with red light is known to have the effect of enhancing the healing effect of the skin and relieving pain and inflammation.

Furthermore, blue light has the effect of removing staphylococci and bacteria, thereby treating inflammation and rashes.

The eye treatment device of Patent Document 1 is characterized in that light is irradiated to the eye for treatment while the chin is supported on a chin rest and the eye area is in close contact with an interface surface.

However, the eye treatment device of Patent Document 1 has a very inconvenient problem for irradiating the eyes for a relatively long time, as it is configured to irradiate light to the eyes while the chin is supported, although there is no problem for a short period.

Patent Document 2 is an eye disease treatment device that treats eye disease by irradiating light to the eyes while being worn on the head. However,

Patent Document 2 has problems in that the structure of the optical system is complex, making manufacturing difficult, and the head-wearing structure is uncomfortable to wear and its heavy weight causes discomfort.
(Patent Document 1) KR 10-2021-0112072 A
(Patent Document 2) KR 10-2017-0058958 A

### DETAILED DESCRIPTION OF THE INVENTION

### Technical Problem

An object of the present invention is to provide a treatment system for eye disease treatment, an eye disease treatment method, and a light module used therein, which are easy to manufacture and convenient to use by simplifying the structure of the light module for light irradiation to the eyes, in view of the above-mentioned problems and trends.

### Technical Solution

The present invention has been created to achieve the above object, and the present invention provides a system for eye disease treatment, comprising: a main body module (10) in which a light module (100) for generating treatment light is provided, and which covers the eyes of the user so that the light from the light module (100) irradiates the user's eyes; a wearing module (20) coupled to the main body module (10) and extending to the side of the user's head to be worn by the user; and a camera unit (300) installed in the main body module (10) for acquiring image of the eyes of the user and tracking whether the light irradiated by the light module (100) is stably irradiated to the user's eyes.

The camera unit (300) may include a camera that recognizes at least one of infrared and visible light.

The camera unit (300) may include: an imaging unit (310) for acquiring images of a user's pair of eyes; an optical system (320) for guiding light so that the imaging unit (310) can acquire images of the pair of eyes while the main body module (10) is worn by the user; and an image processing unit for processing the images acquired by the imaging unit (310).

The image processing unit may perform an eye object detection step of searching for an eye object corresponding to the eye in a primary image acquired by the imaging unit (310); an eye object extraction step of extracting the eye object detected in the eye object detection step; and a normality determination step of analyzing the eye object extracted in the eye object extraction step to determine whether the light irradiation is being performed normally.

At least one of the eye object detection step, the eye object extraction step, and the normality determination step may be performed by an artificial intelligence program.

At least one of the eye object detection step and the normality determination step may be performed by an artificial intelligence program.

The treatment light may be a combination of irradiation lights of a plurality of wavelength bands.

When at least one of the irradiation lights is irradiated, the remaining irradiation lights may be in an off state.

When one of the plurality of irradiation lights is irradiated, the remaining irradiation lights may be in an off state.

The plurality of irradiation lights may be controlled on and off for irradiation.

The treatment light may include at least one of a first irradiation light of a blue wavelength band; a second irradiation light of a green wavelength band; a third irradiation light of a red wavelength band; a fourth irradiation light of an ultraviolet wavelength band; and a fifth irradiation light of an infrared wavelength band.

As another aspect of the present invention, the present invention also discloses a system for eye disease treatment or an eye care system, comprising: a main body module (10) in which a light module (100) for generating treatment light is installed, and which covers the eyes of the user so that the light from the light module (100) irradiates the user's eyes; and a wearing module (20) coupled to the main body module (10) and extending to the side of the user's head to be worn by the user.

The light module (100) may include: an LED module (100) including an LED substrate (112) on which one or more LED devices (111) are installed; a barrel (120) to one end of which the LED module (100) is coupled; one or more lenses (141) installed in the barrel (120) for diverging the light generated by the LED device (111); a diffusion member (142) coupled to the barrel (120) for diffusing the light; and a fixing coupling part (130) coupled to the barrel (120) for fixing the lens (141) and the diffusion member (142).

The present invention also discloses a light module for an eye disease treatment system, comprising: an LED module (100) including an LED substrate (112) on which one or more LED devices (111) are installed; a barrel (120) to one end of which the LED module (100) is coupled; one or more lenses (141) installed in the barrel (120) for diverging the light generated by the LED device (111); a diffusion member (142) coupled to the barrel (120) for diffusing the light; and a fixing coupling part (130) coupled to the barrel (120) for fixing the lens (141) and the diffusion member (142).

The present invention also discloses a light module for an eye disease treatment system, characterized by comprising: a barrel (120) having a hollow cylinder structure; an LED module (110) coupled to one end of the barrel (120), having a through-opening (113) corresponding to the hollow of the barrel (120), and including an LED substrate (112) on which a plurality of LED devices (111) are installed along the circumferential direction; a reflection member (151) coupled to one end of the barrel (120) for reflecting the light generated from the plurality of LED devices (111) toward the hollow of the barrel (120); a spherical lens (152) installed in the barrel (120) for converging the light reflected by the reflection member (151); and a guide lens (153) for guiding the light that has passed through the spherical lens (152) into a preset area.

The LED device (111) may include at least one of a first LED device for irradiating light of a blue wavelength band; a second LED device for irradiating light of a green wavelength band; a third LED device for irradiating light of a red wavelength band; a fourth LED device for irradiating light of an ultraviolet wavelength band; and a fifth LED device for irradiating light of an infrared wavelength band.

The present invention also discloses a system for eye disease treatment that generates three or more lights with different wavelengths to irradiate a user's eyes, wherein the system is a system for eye disease treatment having the above-described configuration, and wherein the three or more lights include lights of a second wavelength of 680 nm±10 nm, a third wavelength of 780 nm±10 nm, and a fourth wavelength of 830 nm±10 nm.

The present invention also discloses a system for eye disease treatment, comprising a light module (100) that generates three or more lights with different wavelengths to irradiate a user's eyes, wherein the three or more lights include lights of a second wavelength of 680 nm±10 nm, a third wavelength of 780 nm±10 nm, and a fourth wavelength of 830 nm±10 nm.

The sum of the irradiation intensity of the second to fourth wavelength lights may be preferably 35-40 mW/cm².

More preferably, the sum of the irradiation intensity of the second to fourth wavelength lights may be 37.8 mW/cm².

It is preferable that the average value of the light irradiation intensity of the second wavelength light is 12.3 mW/cm², the average value of the light irradiation intensity of the third wavelength light is 14.9 mW/cm², and the average value of the light irradiation intensity of the fourth wavelength light is 10.6 mW/cm².

The second to fourth wavelength lights may be simultaneously irradiated to the user's eyes.

The energy of the second to fourth wavelength lights irradiated for 2 minutes may be irradiated to be less than 5.0 J/cm².

The three or more lights may further include a light of a first wavelength of 590±10 nm.

The system for eye disease treatment according to the present invention can be used for the treatment or symptom relief of at least one eye disease among dry eye syndrome, macular edema, and macular degeneration.

### Effects of the Invention

The treatment system for eye disease treatment, eye disease treatment method, and light module used therein according to the present invention have the advantage of being convenient to manufacture due to a simple structure, as a light module corresponding to each of the user's eyes is installed to irradiate each eye with light.

In particular, the treatment system for eye disease treatment, eye disease treatment method, and light module used therein according to the present invention have the advantage of preventing the irradiation of light with excessive intensity to the user's eyes by diverging the light generated from the LED device using a barrel and transmitting the diverged light through a diffusion plate.

In addition, the treatment system for eye disease treatment, eye disease treatment method, and light module used therein according to the present invention have the advantage of being convenient to manufacture due to a simple overall structure, by using a reflection member for the light path from the LED device to the user's eyes when irradiating each eye with light, as a light module corresponding to each of the user's eyes is installed.

According to another aspect of the present invention, the treatment system for eye disease treatment, eye disease treatment method, and light module used therein according to the present invention have the advantage of being able to treat and alleviate eye diseases such as dry eye syndrome, macular degeneration, and macular edema.

### BRIEF DESCRIPTION OF THE DRAWINGS

FIG. 1 is a perspective view showing an example of a system for treating an eye disease according to the present invention.

FIG. 2A is a perspective view of the system for treating an eye disease of FIG. 1 viewed from another angle, and FIGS. 2B and 2C are cross-sectional views of the system for treating an eye disease of FIG. 1 at different horizontal heights, respectively.

FIG. 2D is a cross-sectional view taken along the line D-D in FIG. 2C.

FIG. 2E is an exploded perspective view showing a part of the camera unit shown in FIGS. 2C to 2D.

FIG. 3 is a perspective view showing a state where a cover part is removed from the system for treating an eye disease of FIG. 1.

FIG. 4 is a front view of FIG. 3, and FIG. 5 is a rear view of FIG. 3.

FIG. 6A is a perspective view of a light module of the system for treating an eye disease of FIG. 1, and FIG. 6B is an exploded perspective view of the light module of the system for treating an eye disease of FIG. 1.

FIG. 7A is a cross-sectional view of the light module of FIG. 6A, and FIG. 7B is a cross-sectional view according to a modified example of the light module of FIG. 7A.

FIG. 8 is a front view of an LED module of the light module of FIG. 6A.

FIG. 9 is a perspective view showing a state where the system for treating an eye disease shown in FIG. 2 is worn.

FIG. 10 is a cross-sectional view showing another example of a light module of the system for treating an eye disease according to the present invention.

FIG. 11 is a perspective view showing a modified example of the light module of the system for treating an eye disease shown in FIG. 10.

FIG. 12 is an exploded perspective view of the system for treating an eye disease of FIG. 11.

FIG. 13 is a plan view showing an LED module of the system for treating an eye disease of FIG. 11.

FIG. 14 is a longitudinal sectional view of the system for treating an eye disease of FIG. 11. FIG. 15A is a graph showing the eye treatment monitoring performed by the image processing unit, and FIG. 15B shows a plurality of irradiation lights which are irradiated such that when one of the plurality of irradiation lights is irradiated, the remaining irradiation lights are in an off state.

FIG. 16 is a photograph showing a simply configured test apparatus for verifying the light irradiation effect of the eye care device according to the present invention.

FIG. 17 is a graph showing the test process by the test apparatus shown in FIG. 16 in chronological order.

FIG. 18 is a photograph showing human corneal epithelial cells for evaluating the safety of light irradiation by the test apparatus shown in FIG. 16.

FIG. 19 are graphs showing the change in survival rate and proliferation of human corneal epithelial cells according to the light irradiation time by the test apparatus shown in FIG. 16.

FIG. 20 is a graph showing the amount of tears measured by multi-wavelength LED irradiation in an animal model of dry eye.

FIG. 21 shows photographs and a graph showing the results of corneal surface irregularity evaluation by multi-wavelength LED irradiation in an animal model of dry eye.

FIG. 22 shows photographs and a graph showing the results of evaluating the detached corneal surface density and area by multi-wavelength LED irradiation in an animal model of dry eye.

FIG. 23 shows photographs and a graph showing the results of corneal thickness evaluation by multi-wavelength LED irradiation in an animal model of dry eye.

FIG. 24 shows photographs and a graph showing the results of IL-1β expression analysis by multi-wavelength LED irradiation in an animal model of dry eye.

FIG. 25 shows photographs and a graph showing the results of IL-6 expression analysis by multi-wavelength LED irradiation in an animal model of dry eye.

FIG. 26 shows photographs and a graph showing the results of TNFα expression analysis by multi-wavelength LED irradiation in an animal model of dry eye.

FIG. 27 shows photographs and a graph showing the change in cell survival rate of retinal pigment epithelial cells according to multi-wavelength LED irradiation time.

FIG. 28 shows photographs and a graph showing the change in proliferation of retinal pigment epithelial cells according to multi-wavelength LED irradiation time.

FIG. 29 shows photographs and a graph showing the establishment of a dry AMD animal model condition over time after sodium iodide treatment.

FIG. 30 shows photographs and a graph comparing the ONL thickness in the retina after multi-wavelength LED treatment in a macular degeneration animal model.

FIG. 31 shows photographs and a graph showing the results of a TUNEL assay after multi-wavelength LED treatment in a macular degeneration animal model.

FIG. 32 shows photographs and a graph showing the results of PKCα-positive cells in the retina after multi-wavelength LED treatment in a macular degeneration animal model.

FIG. 33 shows photographs and a graph showing the results of fluorescence expression intensity analysis of Rhodopsin and RPE65 after multi-wavelength LED treatment in a macular degeneration animal model.

FIG. 34 shows photographs of (A) STZ administration for inducing diabetic retinopathy (macular edema), (B) multi-wavelength LED irradiation, (C) blood glucose measurement results of the normal control group, and (D) blood glucose measurement results of the diabetic retinopathy (macular edema) model.

FIG. 35 is a graph showing the body weight change of the normal control group and the diabetic retinopathy (macular edema) animal model after STZ administration.

FIG. 36 shows photographs and a graph comparing the ONL thickness in the retina after multi-wavelength LED treatment in the diabetic retinopathy (macular edema) animal model.

### BEST MODE FOR CARRYING OUT THE INVENTION

Hereinafter, a treatment system for eye disease treatment, an eye disease treatment method, and a light module used therein according to the present invention will be described with reference to the accompanying drawings.

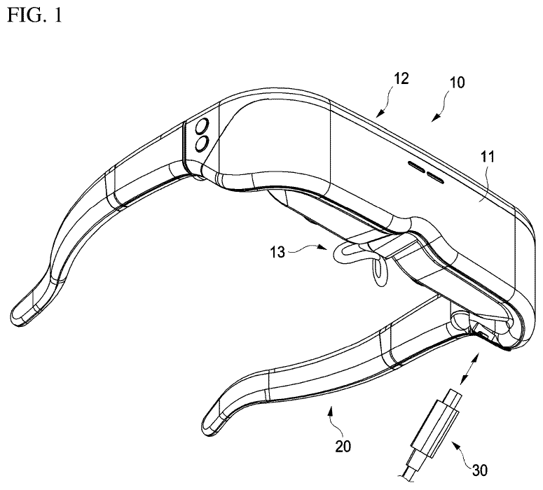

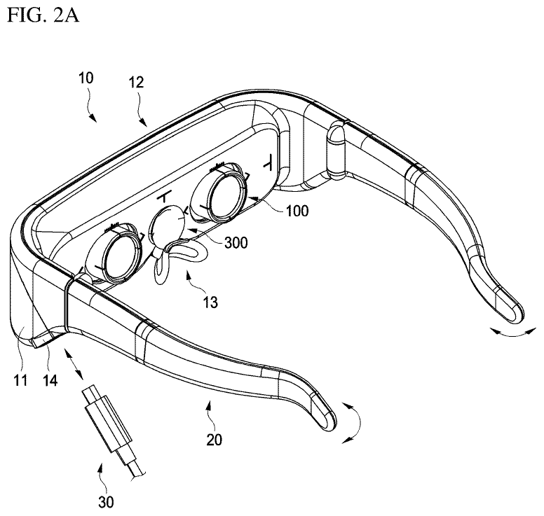

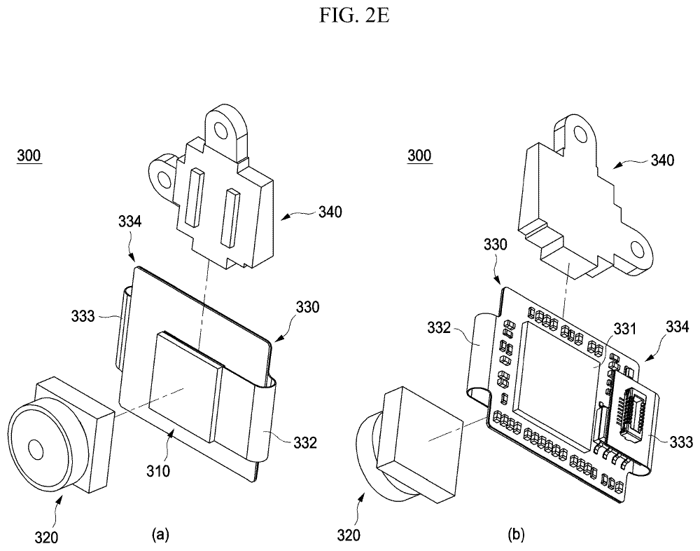

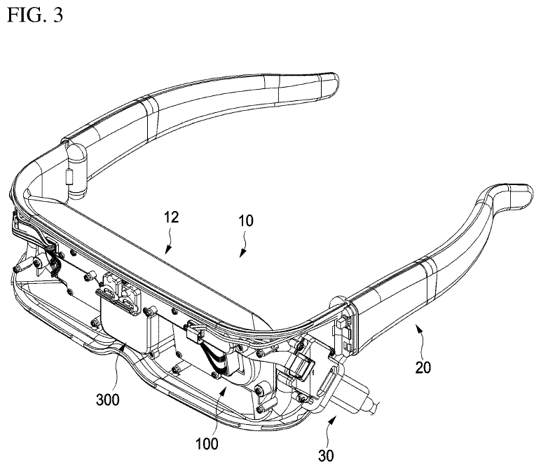

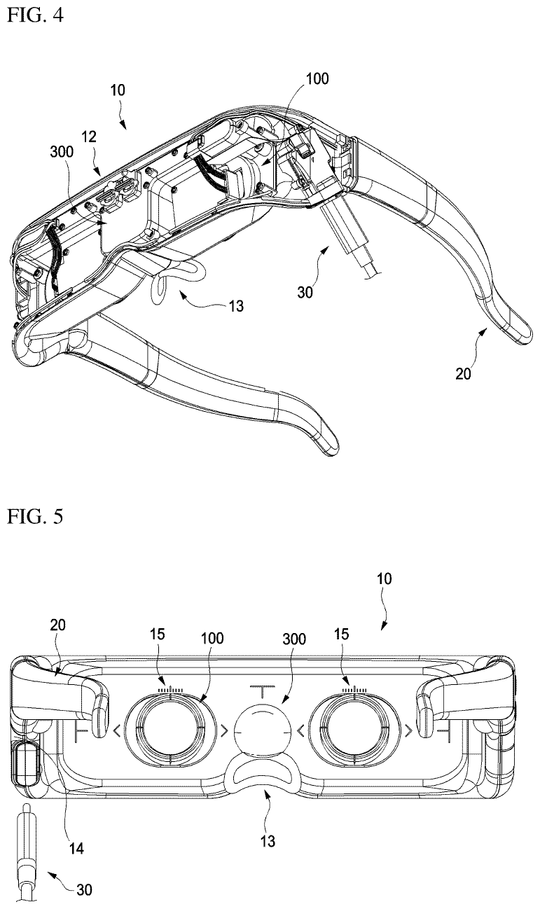

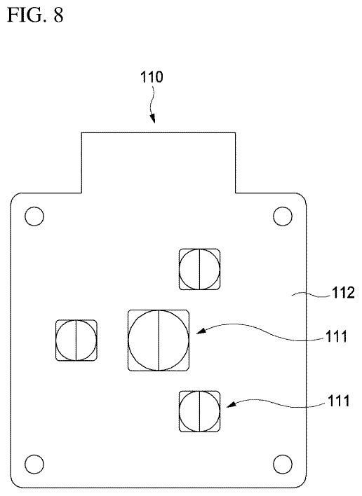

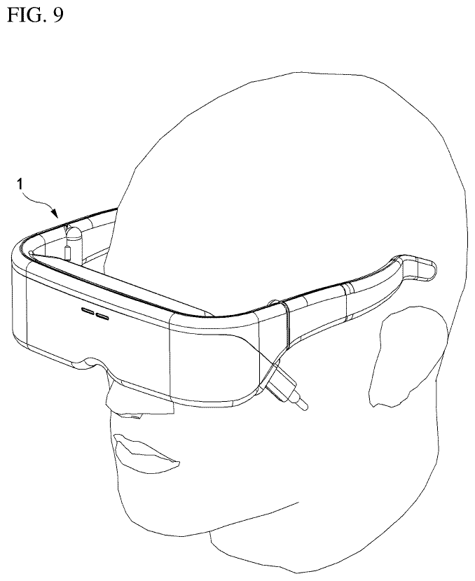

As shown in FIGS. 1 to 9, the system for eye disease treatment according to the present invention includes: a main body module (10) in which a light module (100) for generating treatment light is installed, and which covers the eyes of the user so that the light from the light module (100) irradiates the user's eyes; and a wearing module (20) coupled to the main body module (10) and extending to the side of the user's head to be worn by the user.

The main body module (10) is a component in which the light module (100) for generating treatment light is installed and which covers the user's eyes so that the light from the light module (100) irradiates the user's eyes, and various configurations are possible.

Here, the light irradiated to the user's eyes may be irradiated to at least one of the user's cornea and retina.

Therefore, it is preferable that the light irradiated to the user's eyes is guided by an optical system to be irradiated to the user's cornea and/or retina.

At this time, when irradiating the user's eyes for the care, treatment, or symptom relief of the user's retina, it is preferable that the light irradiation intensity and light irradiation conditions (irradiation time, irradiation pattern, etc.) are performed to an extent that does not damage the user's retina.

For example, the main body module (10) may include a main body part (12) to which the light module (100) is coupled and a later-described wearing module (20) is coupled, and a cover part (11) coupled to the main body part (12) to cover the light module (100).

The main body part (12) is a component to which the light module (100) is coupled and a later-described wearing module (20) is coupled, and various configurations are possible depending on the coupling structure with the light module (100) and the nose support structure.

The main body part (12) may have a shape that crosses both eyes based on the user's face, with a control unit (not shown) for controlling the light module (100) built inside and the light module (100) coupled to the lower side.

The control unit is a component for controlling the light module (100) inside, and may be composed of one or more circuit boards.

Meanwhile, the control unit may include a communication module for communication with the outside.

The communication module is a module for communicating with the outside by at least one of wired and wireless method, and various configurations are possible depending on the communication method.

For example, the communication module may include at least one communication module such as Bluetooth, Wi-Fi, Zigbee, etc.

In addition, the communication module may be installed in the main body module (10) or in the wearing module (20).

Meanwhile, light modules may be coupled to the lower side on the left and right sides, respectively, corresponding to the user's pair of eyes.

In addition, a nose pad part (13) may be coupled between the pair of light modules (100).

The wearing module (20) is a component that is coupled to the main body module (10) and extends to the side of the user's head to be worn by the user, and various configurations are possible depending on the wearing structure.

As an example, the wearing module (20) may be configured as a pair extending from both ends of the main body module (10) toward the user's ears.

And the end portion of the wearing module (20) may have a structure similar to glasses so that it can be hooked on the user's ears.

Meanwhile, considering that the user's head size, distance to the ears, etc., are various, the wearing module (20) may be coupled to the main body module (10) such that its coupling state, for example, its length from the main body module (10) is variable, or it may be hinge-rotatably coupled to be foldable with respect to the main body module (10), thus having various coupling structures.

That is, the wearing module (20) may be hinge-coupled by a hinge part (21) at both ends of the main body module (10) for convenience of storage, etc., so that it can be folded and stored with respect to the main body module (20) when not in use.

And at least one of the main body module (10) and the wearing module (20) may be provided with a terminal part (14) for connection with an external connection terminal (30).

The terminal part (14) is a component installed on at least one of the main body module (10) and the wearing module (20) for connection with the external connection terminal (30), and various configurations are possible depending on the connection structure with the external connection terminal (30).

In addition, although not shown, a battery (40) for supplying power to the light module (100) and the control unit may be built into the wearing module (20).

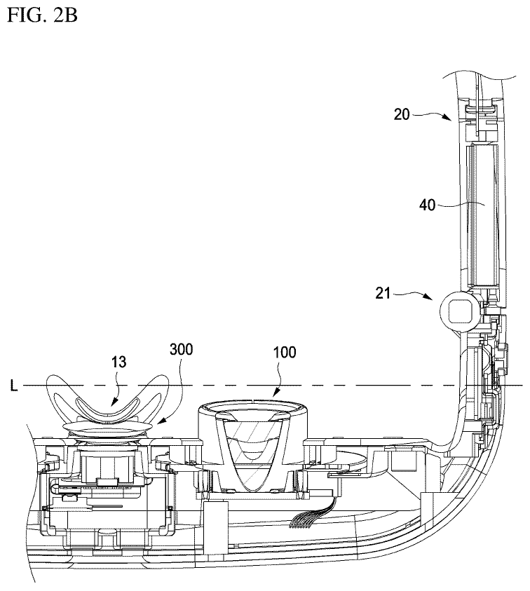

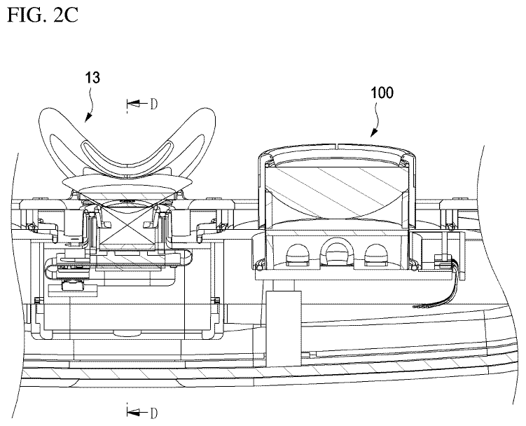

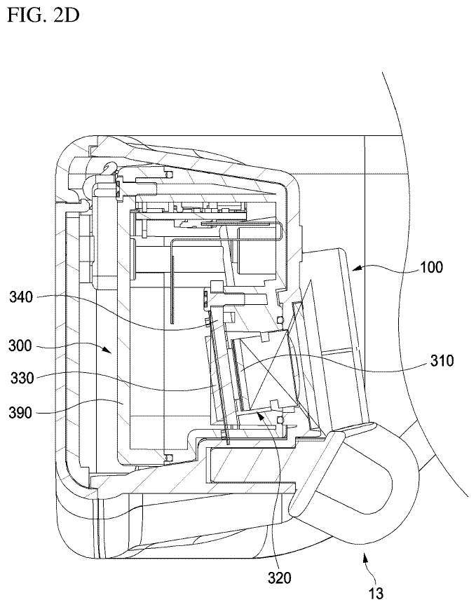

As shown in FIG. 2B, the battery (40) can be installed in the wearing module (20), thereby making the overall weight distribution uniform.

That is, the battery (40) may be a rechargeable battery such as a lithium-ion battery and may be installed inside extending long along the length direction of the wearing module (20).

Meanwhile, the battery (40) may be installed in the main body module (10) in addition to or along with being installed in the wearing module (20), of course.

The light module (100) is a component that generates treatment light, and any configuration is possible as long as it can be coupled to the main body module (10).

For example, the light module (100) may be configured to include an LED module (110) that irradiates treatment light, and an optical system (101) that guides the light irradiated from the LED module (120).

Here, the light module (100) is coupled to the main body module (10) by a coupling means (102).

The coupling means (102) is a component for coupling the light module (100) to the main body module (10), and various configurations are possible.

For example, the coupling means (102) may be configured as a part of a housing (not shown) in which the light module (100) is installed.

In addition, the coupling means (102) may be configured as a coupling part (not shown) to which the light module (100) is coupled, as a partial configuration of the main body module (10), particularly the main body part (12).

Meanwhile, since the distance between the user's two eyes may vary from person to person, it is necessary to adjust the light module (100) to the distance between the user's eyes.

Accordingly, the coupling means (102) may be configured such that the distance formed by the pair of light modules (100), the so-called pupillary distance (PD), is variable according to the distance between the user's eyes.

At this time, display scale (15) for indicating a position based on the pupillary distance (PD) may be provided on the main body part (12) of the main body module (10).

In addition, depending on the user's wearing state, it may be necessary to align the direction of the light module (100) facing the eyes, that is, the axial direction, with the eyes.

Accordingly, the coupling means may be configured such that the axial direction of the light module (100) is variable in a state of being worn on the user's head.

The axial direction variable structure may be a structure capable of rotating on one or more axes, and may be configured as a universal joint structure or a structure capable of rotating around at least one of the X, Y, and Z axes.

The LED module (110) is a component for irradiating treatment light, and various configurations are possible.

For example, the LED module (110) may include an LED substrate (112) on which one or more LED devices (111) are installed.

The LED device (111) is a component that generates treatment light, and the wavelength band can be selected according to the optical characteristics of the care light.

In particular, since it is preferable to irradiate light of a plurality of wavelength bands to maximize the care (treatment) effect, it is preferable that a plurality of LED devices (111) are configured.

For example, the LED device (111) may include at least one of a first LED device for irradiating light of a blue wavelength band; a second LED device for irradiating light of a green wavelength band; a third LED device for irradiating light of a red wavelength band; a fourth LED device for irradiating light of an ultraviolet wavelength band; and a fifth LED device for irradiating light of an infrared wavelength band.

The first LED device is an LED device that irradiates light of a blue wavelength band, and an LED device that irradiates light of an appropriate wavelength can be selected according to the level and content of care for the irradiated body part.

For example, the first LED device may be an LED device that irradiates light with a wavelength of 435-485 nm.

The second LED device may be an LED device that irradiates light of a green wavelength band.

For example, the second LED device may be an LED device that irradiates light with a wavelength of 500-600 nm, preferably 500-590 nm, and more preferably 500-560 nm.

Meanwhile, considering commercially available LED devices, the second LED device may be an LED device that irradiates light with a wavelength of 590±10 nm.

The third LED device may be an LED device that irradiates light of a red wavelength band.

For example, the third LED device may be an LED device that irradiates light with a wavelength of 610-760 nm, particularly 680±10 nm.

The fourth LED device may be an LED device that irradiates light of an ultraviolet wavelength band.

The fourth LED device may be an LED device that irradiates light with wavelengths of UVA (320-400 nm), UVB (290-320 nm), and UVC (200-0 nm).

Meanwhile, if the fourth LED device is used for purposes such as care rather than treatment, it can also be used for disinfecting personal belongings.

The fifth LED device is an LED device that irradiates light of an infrared wavelength band, and an LED device that irradiates light of an appropriate wavelength can be selected according to the level and content of care for the irradiated body part.

The fifth LED device may be an LED device that irradiates light with a wavelength of 750 nm to 25,000 nm. Here, the infrared light may be classified into near-infrared (750 nm to 1,500 nm), mid-infrared (1,500 nm to 3,000 nm), and far-infrared (3,000 nm to 25,000 nm) according to the wavelength range.

Meanwhile, for the first to fifth LED devices as described above, only one type of LED device may be used to irradiate a single light, or a combination of at least two types of LED devices from the first to fifth LED devices may be used.

For example, the LED module (100) may be configured by a combination of a first LED device, a second LED device, and a third LED device.

As another example, the LED module (100) may be configured by a combination of a first LED device, a second LED device, and a third LED device.

In addition, the LED module (100) may be composed of only the fifth LED device, which emits light of two or more wavelengths within the same wavelength series, for example, an LED device that irradiates infrared light of 780±10 nm and an LED device that irradiates infrared light of 830±10 nm may be used.

That is, the light combination module for a system for eye disease treatment according to the present invention may be composed of two or more types of LED devices with different wavelengths, depending on the use and purpose.

Meanwhile, as a specific embodiment of the combination of the LED devices (111), it may be configured by a combination of a first LED device with a wavelength of 590±10 nm, a second LED device with a wavelength of 680±10 nm, a third LED device with a wavelength of 780±10 nm, and a fourth LED device with a wavelength of 830±10 nm.

In summary, the treatment light for eye disease treatment can be a combination of irradiation lights of a plurality of wavelength bands.

For example, the treatment light may include: a first irradiation light of a blue wavelength band; a second irradiation light of a green wavelength band; a third irradiation light of a red wavelength band; a fourth irradiation light of an ultraviolet wavelength band; and at least one of a fifth irradiation light of an infrared wavelength band.

As another example, the treatment light may be configured by a combination of a first light with a wavelength of 590±10 nm, a second light with a wavelength of 680±10 nm, a third light with a wavelength of 780±10 nm, and a fourth light with a wavelength of 830±10 nm.

Meanwhile, if the irradiation energy of the light irradiated to the user's eyes is excessive, retinal cells may be damaged.

Therefore, it is preferable that the total amount of irradiation energy per hour of the treatment light irradiated to the user's eyes is equal to or less than a preset value.

Therefore, to reduce power consumption for phototherapy and to exclude light interference when combining treatment lights, it is preferable that when at least one of the treatment lights, that is, the irradiation lights, is irradiated, the remaining irradiation lights are in an off state.

As shown in FIG. 15b, it is more preferable that when one of the plurality of irradiation lights is irradiated, the remaining irradiation lights are in an off state.

Specifically, after the first irradiation light (wavelength λ1) is irradiated for a preset first period, the second irradiation light (wavelength λ2) may be irradiated for a second period, the third irradiation light (wavelength λ3) for a third period, and the fourth irradiation light (wavelength λ4) for a fourth period, sequentially.

Here, the order and period of the wavelengths of the irradiation light (λ1, λ2, λ3, λ4) can be set in various ways.

In addition, the power value of the irradiation light can also be set in various ways.

Meanwhile, it is preferable that the plurality of irradiation lights are pulse lights and are irradiated with on-off control.

When the treatment light is irradiated to the user's eyes in the above pattern, it is possible to minimize power consumption and at the same time exclude interference due to overlapping of the irradiation lights to perform light irradiation in an optimal state.

Meanwhile, in the irradiation of the treatment light described above, the type of irradiation light (based on wavelength), the number of irradiation lights, and the power value of each irradiation light (in mW units, etc.) can be set in various ways.

If the type of irradiation light (based on wavelength), the number of irradiation lights, and the power value of each irradiation light (in mW units, etc.) can be set in various ways, it is possible to provide an optimized light irradiation effect for each user or for each type of eye disease.

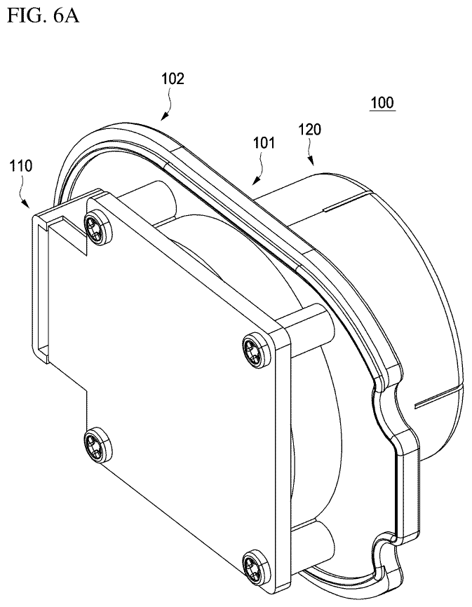

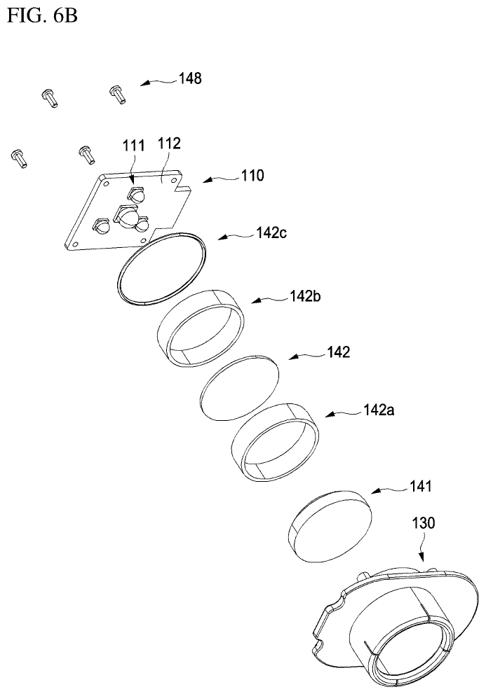

Meanwhile, as an integrated configuration, the light module (100) according to the first embodiment may, as shown in FIGS. 6 to 8, include an LED module (100) including an LED substrate (112) on which one or more LED devices (111) are installed; a barrel (120) to one end of which the LED module (100) is coupled; one or more lenses (141) installed in the barrel (120) for diverging the light generated by the LED device (111); a diffusion member (142) coupled to the barrel (120) for diffusing light; and a fixing coupling part (130) coupled to the barrel (120) for fixing the lens (141) and the diffusion member (142).

The barrel (120) is a component to one end of which the LED module (100) is coupled, and various configurations are possible.

For example, the barrel (120) may have a hollow cylinder structure with both ends open.

In addition, the inner circumferential surface of the barrel (120) may form a reflective surface so that the light generated from the LED module (100) is reflected.

The lens (141) is a component installed in the barrel (120) for diverging the light generated by the LED device (111), and one or more may be installed.

For example, the lens (141) may be a convex lens, configured as a plano-concave lens.

At this time, the flat surface of the lens (141) may be installed to face the LED module (100).

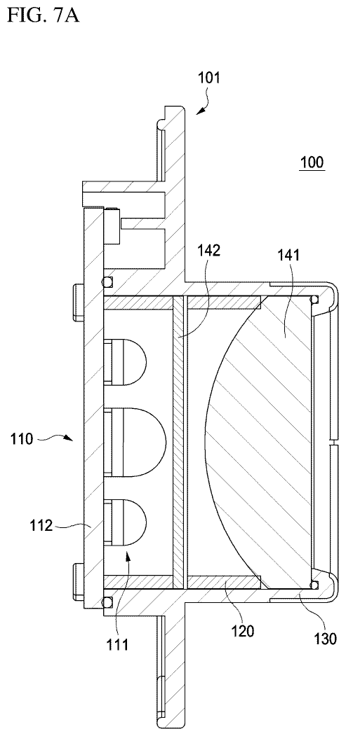

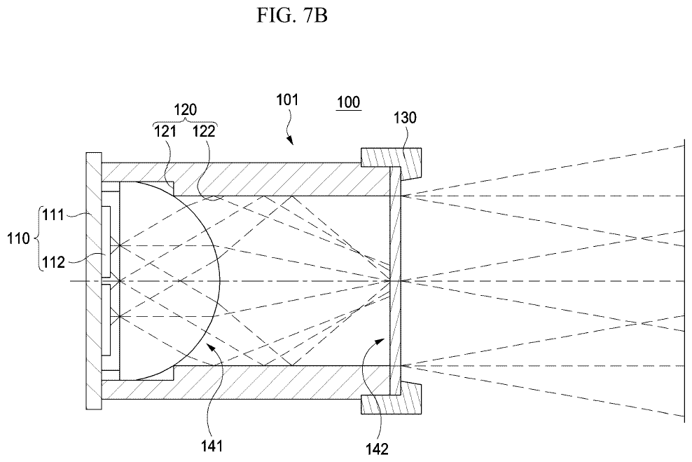

Meanwhile, the barrel (120) can have various structures depending on the coupling structure with the lens (141) and the diffusion member (142), and as shown in FIG. 7B, it may be composed of a large-diameter part (121) having an inner diameter corresponding to the outer peripheral surface of the edge of the lens (141), and a small-diameter part (122) extending from the large-diameter part (121) and having a smaller inner diameter than the large-diameter part (121).

At this time, the boundary portion between the large-diameter part (121) and the small-diameter part (122) may be installed to form a step and support the outer peripheral surface of the lens (131).

The diffusion member (142) is a component coupled to the other end of the barrel (120) for diffusing light, and various configurations are possible.

Specifically, since the intensity of the light generated from the LED device (111) is strong and can cause fatigue to the user's eyes, the diffusion member (142) is a diffusion member for diffusing light to prevent this, and various diffusion members can be used depending on the type of diffusion.

Meanwhile, one or more auxiliary cylinders (142a, 142b) may be installed inside the barrel (120) for the installation of the diffusion member (142).

And the barrel (120) may be provided with a sealing member (142c) such as an O-ring for sealing at the surface coupled with the LED substrate (112).

The fixing coupling part (130) is a component coupled to the other end of the barrel (120) to fix the diffusion member (142), and various configurations are possible.

For example, as shown in FIG. 7B, the fixing coupling part (130) may include a rim part (131) having a female screw part formed to be screw-coupled to a male screw part formed on the outer circumferential surface of the barrel (120) and pressing the diffusion member (142) against the end of the barrel (120) on the inner side.

Meanwhile, the optical system (101) can have various configurations depending on the irradiation direction of the treatment light, etc.

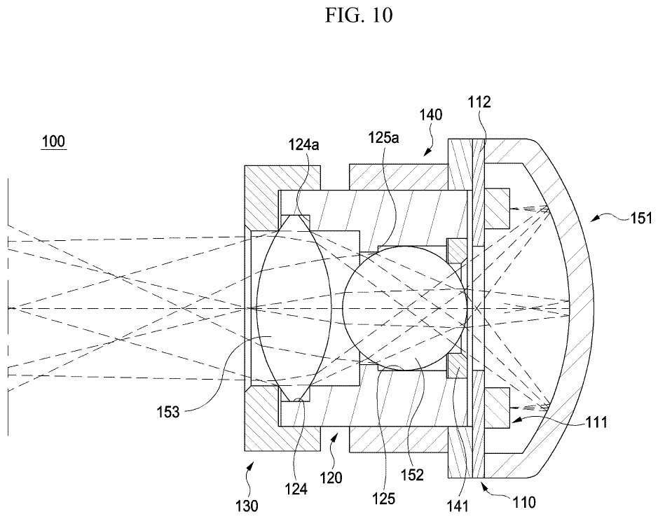

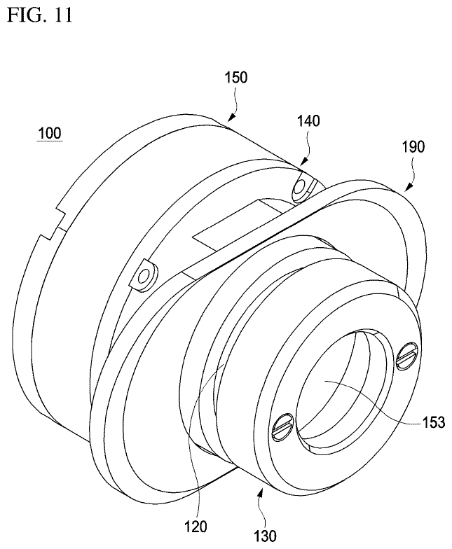

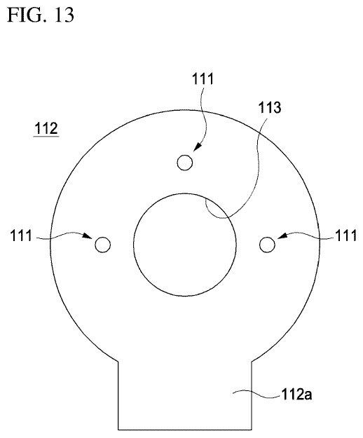

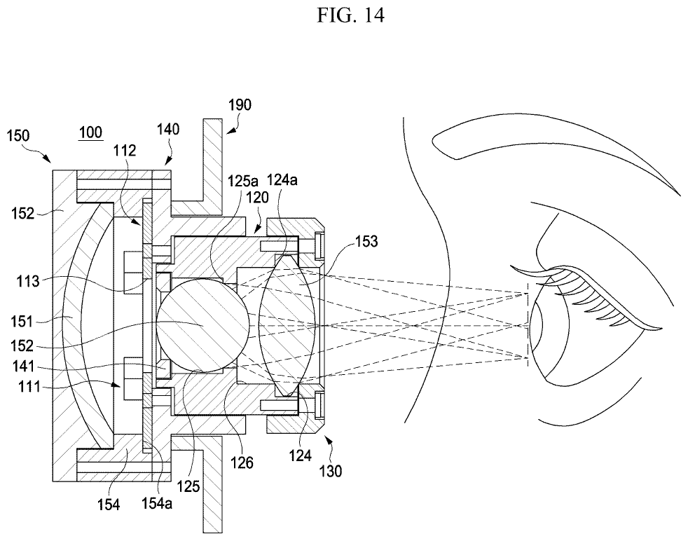

For example, as shown in FIGS. 10 to 14, the light module (100) according to the present invention includes a barrel (120) having a hollow cylinder structure; an LED module (110) coupled to one end of the barrel (120), having a through-opening (113) corresponding to the hollow of the barrel (120), and including an LED substrate (112) on which a plurality of LED devices (111) are installed along the circumferential direction; a reflection member (151) coupled to one end of the barrel (120) for reflecting the light generated from the plurality of LED devices (111) toward the hollow of the barrel (120); a spherical lens (152) installed in the barrel (120) for converging the light reflected by the reflection member (151); and a guide lens (153) for guiding the light that has passed through the spherical lens (152) into a preset area.

The barrel (120) is a component having a hollow cylinder structure, and various configurations are possible.

For example, the barrel (120) may include a large-diameter part (124) having an inner diameter corresponding to the outer diameter of a later-described guide lens (153), and a small-diameter part (125) having an inner diameter corresponding to the outer diameter of the spherical lens (152).

The large-diameter part (124) is a part having an inner diameter corresponding to the outer diameter of a later-described guide lens (153), and may have an inner diameter corresponding to the outer diameter part of the guide lens (153) so that the guide lens (153) can be precisely installed.

Meanwhile, the large-diameter part (124) may be formed with a stepped part (124a) that prevents the axial movement of the guide lens (153) to prevent axial movement.

By the formation of the stepped part (124a), a medium-diameter part (126) may be formed between the large-diameter part (124) and the small-diameter part (125) in the barrel (120).

The small-diameter part (125) is a part having an inner diameter corresponding to the outer diameter of the spherical lens (152), and may have an inner diameter corresponding to the outer peripheral surface of the spherical lens (152) so that the spherical lens (152) can be precisely installed.

At this time, the small-diameter part (125) may be formed with a locking jaw (125a) to prevent the axial movement of the spherical lens (152).

And a pressing member (141) for pressing the spherical lens (152) in the axial direction may be installed on the opposite side of the small-diameter part (125) where the locking jaw (125a) is formed.

The pressing member (140) is installed in the small-diameter part (125) and presses the spherical lens (152) in the axial direction from the side opposite to where the locking jaw (125a) is formed, and an opening is formed in the central part to allow the passage of light.

Meanwhile, the pressing member (140) may be pressed in the direction toward the spherical lens (152) by the LED substrate (112) when the LED module (110) is directly or indirectly coupled to the barrel (120).

At this time, it is preferable that the pressing member (140) has a material capable of elastic deformation, such as synthetic resin or rubber, for close contact with the spherical lens (152).

The LED module (110) is a component coupled to one end of the barrel (120), having a through-opening (113) corresponding to the hollow of the barrel (120), and including an LED substrate (112) on which a plurality of LED devices (111) are installed along the circumferential direction, and various configurations are possible.

The LED device (111) is the same as or similar to the previously described LED device (111), so a detailed description thereof is omitted.

The LED substrate (112) is coupled to one end of the barrel (120), a through-opening (113) corresponding to the hollow of the barrel (120) is formed, and a plurality of LED devices (111) are installed along the circumferential direction.

The through-opening (113) is formed with a sufficient size to allow the transmission of light reflected by a later-described reflection member (151) from the LED substrate (112).

Meanwhile, the LED devices (111) installed on the LED substrate (112) are installed to irradiate light in the direction opposite to the barrel (120).

The reflection member (151) is a component coupled to one end of the barrel (120) for reflecting the light generated from the plurality of LED devices (111) toward the hollow of the barrel (120), and various configurations are possible.

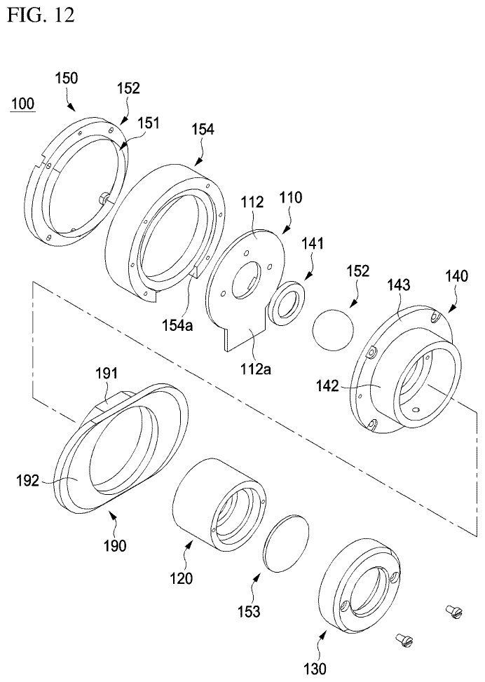

For example, as shown in FIGS. 10 and 12, the inner circumferential surface of the reflection member (151) may be formed with a preset curvature to guide light to the user's eyes.

In addition, a protection member (152) for preventing impact, damage, etc. from the outside may be coupled to the reflection member (151) integrally or as a separate member.

The protection member (152) is a component provided to prevent impact, damage, etc. to the reflection member (151) from the outside, and various configurations are possible, such as being formed integrally with the reflection member (151) or being coupled separately.

Meanwhile, the reflection member (151) and the protection member (152) may be coupled to the barrel (120) by various structures together with the LED module (110) as a single reflection module (150).

The reflection module (150) is a module composed of the reflection member (151) and the protection member (152) for coupling with a later-described coupling member (140) and the LED module (110), and various configurations are possible.

For example, the reflection module (150) may include an auxiliary cylinder (154) that couples the LED substrate (112) to the coupling member (140) when the reflection member (151) and the protection member (152) are coupled to the coupling member (140) by screws or the like.

The auxiliary cylinder (154) is a component that couples the LED substrate (112) to the coupling member (140) when the reflection member (151) and the protection member (152) are coupled to the coupling member (140) by screws or the like, and various configurations are possible.

For example, the auxiliary cylinder (154) may have an inner diameter corresponding to the outer diameter of the reflection member (151) so that the reflection member (151) can be inserted, and a step (154a) corresponding to the thickness of the LED substrate (112) may be formed so that the LED substrate (112) is coupled to the coupling member (140).

In addition, the auxiliary cylinder (154) may be formed with a cutout (154a) that exposes a terminal part (112a) to the outside so that the terminal part (112a) for controlling and supplying power to the LED substrate (112) can be connected to the outside.

Meanwhile, the reflection module (150) and the LED module (110) may be coupled with a coupling member (140) for coupling with the barrel (120).

The coupling member (140) is a component that is coupled to the barrel (120) in a state where the reflection module (150) and the LED module (110) are coupled, and various configurations are possible depending on the coupling structure with the barrel (120).

For example, the coupling member (140) may include a coupling cylinder (142) into which the outer circumferential surface of the barrel (120) is inserted, and a coupling flange (143) extending in the radial direction from the coupling cylinder (142) to which the reflection module (150) and the LED module (110) are coupled.

The spherical lens (152) is a component installed in the barrel (120) for converging the light reflected by the reflection member (151), and various configurations are possible.

The guide lens (153) is a component for guiding the light that has passed through the spherical lens (152) into a preset area, and various configurations are possible.

Meanwhile, the light module (100) having the above-described configuration can be configured as the light module of the system for eye disease treatment shown in FIGS. 1 to 9, and at this time, a coupling part (190) for coupling with the main body module (10) may be coupled for installation into the main body module (10).

The coupling part (190) is a component for coupling with the main body module (10), and various configurations are possible.

For example, the coupling part (190) may include a coupling cylinder (191) into which the barrel (120), particularly the coupling cylinder (142), is inserted and coupled, and a coupling protrusion (192) protruding in the radial direction from the outer circumferential surface of the coupling cylinder (191) and coupled with the main body module (10).

And the coupling cylinder (191) may be coupled with the barrel (120), particularly the coupling cylinder (142), by various structures.

Meanwhile, when the system for eye disease treatment according to the present invention has a structure similar to glasses as shown in FIGS. 1 to 14, it is necessary to confirm whether the light is stably irradiated to the user's eyes in the worn state.

Accordingly, the main body module (10) may be additionally provided with a camera unit (300) for confirming whether the light is stably irradiated to the user's eyes.

The camera unit (300) is a component for acquiring image of the eyes of the user and tracking whether the light irradiated by the previously described light module (100) is stably irradiated to the user's eyes, and may include an imaging device (not shown) and an optical system for forming an image including the user's eyes on the imaging device.

In particular, when the system for eye disease treatment according to the present invention has a structure similar to glasses as shown in FIGS. 1 to 14, the camera unit (300) is installed between the pair of light modules (100) and configured to enable wide-angle shooting such as with a fisheye lens, thereby allowing it to acquire images of the user's pair of eyes.

Meanwhile, considering that the system for eye disease treatment according to the present invention is worn in close contact with the user's face, which may not provide sufficient illumination conditions for acquiring eye images, the camera unit (300) may include a camera that recognizes at least one of infrared and visible light.

In particular, it is preferable that the camera is capable of shooting in the infrared region as well as the visible light region.

Meanwhile, to implement the above functions, the camera unit (300) may, as shown in FIGS. 2a to 2e, include an imaging unit (310) for acquiring images of a user's pair of eyes; an optical system (320) for guiding light so that the imaging unit (310) can acquire images of the pair of eyes while the main body module (10) is worn by the user; and an image processing unit for processing the images acquired by the imaging unit (310).

The imaging unit (310) is a component for acquiring images of a user's pair of eyes, and may be configured as an imaging module equipped with a CCD image sensor, a CMOS image sensor, or the like.

The optical system (320) is a component for guiding light so that the imaging unit (310) can acquire images of the pair of eyes while the main body module (10) is worn by the user, and it is necessary to maintain a preset distance from the user's glabella so that both eyes can be photographed by one camera.

Accordingly, it is preferable that the optical system (320) is located at the user's glabella based on the state where the main body module (10) is worn by the user, and is set to be 15 mm to 40 mm from the user's retina (approximately indicated by Lin FIG. 2B).

For reference, considering that the distance between the objective lens of the light module (100) and the cornea is preferably 1.5 cm to 2.0 cm, particularly 1.7 cm, it is preferable that the distance between the objective lens of the optical system (320) and the cornea is set to be larger than the distance between the objective lens of the light module (100) and the cornea.

For example, the distance between the objective lens of the optical system (320) and the cornea may be set to be 5 mm to 15 mm larger than the distance between the objective lens of the light module (100) and the cornea.

And it is preferable that the optical system (320) has a viewing angle of 120° or more based on the image formed on the imaging unit (310).

Meanwhile, the optical system (320) and the imaging unit (310) may be coupled to each other by a fixing member (340) as shown in FIGS. 2c to 2e.

The fixing member (340) is a component that is coupled to a camera housing (390) to press from the rear side of the imaging unit (310) toward the optical system (320), and various configurations are possible.

The image processing unit is a component for processing the image acquired by the imaging unit (310), and various configurations are possible depending on the processing level of the image captured by the imaging unit (310).

As an example, the image processing unit may be configured to simply process image data so that image data such as raw data files or compressed files like the jpeg format can be transmitted to a separate control unit.

As another example, the image processing unit may include an image processing chip (331) for fast image processing and analysis.

The image processing chip (331) can be any chip that can perform processing of the image acquired by the imaging unit (310) and analysis of the processed image.

And the image processing chip (331) may be mounted on a PCB part (330), and the PCB part (330) may be fixed and installed on the rear surface of the previously described fixing member (340).

And the PCB part (330) may be connected to the imaging unit (310) by a flexible cable (332).

In addition, the PCB part (330) may be connected to a main PCB by a flexible cable (333) for controlling the image processing chip (331), controlling the imaging unit (310), and so on.

In particular, by connecting the flexible cable (332) that connects the imaging unit (310) and the PCB part (330) in a U-shape around the fixing member (240), the space where the imaging unit (310) and the PCB part (330) are installed can be minimized.

That is, the imaging unit (310) and the PCB part (330) are fixed to the front and rear surfaces, respectively, with the fixing member (340) in between, thereby minimizing their installation space.

Meanwhile, the image processing unit, particularly the image processing chip (331), may be configured to perform eye treatment monitoring, as shown in FIG. 15a, which includes an eye object detection step of searching for an eye object corresponding to the eye in a primary image acquired by the imaging unit (310); an eye object extraction step of extracting the eye object detected in the eye object detection step; and a normality determination step of analyzing the eye object extracted in the eye object extraction step to determine whether the light irradiation is being performed normally.

The eye object detection step is a step of searching for an eye object corresponding to the eye in a primary image acquired by the imaging unit (310), and can be performed by various methods.

Here, the primary image is the image itself acquired by the imaging unit (310), and as described above, it can be image data such as raw data files or compressed files like the jpeg format.

The eye object is a region corresponding to the user's eye in the primary image for determining whether the light irradiation is being performed normally, and the detection of the eye object can be performed by various image processing methods.

In particular, the eye object detection step may be performed by an artificial intelligence program trained through various pre-captured eye image data and actual shooting processes.

Here, the eye object detection step, and furthermore the eye treatment monitoring, may be performed based on 30 frames (about 2 seconds) considering the processing speed.

The eye object extraction step is a step of extracting the eye object detected in the eye object detection step for performing a later-described normality determination step, and may be performed by a so-called image cropping method.

The normality determination step is a step of analyzing the eye object extracted in the eye object extraction step to determine whether the light irradiation is being performed normally, and can be performed by various methods.

In particular, the normality determination step may be performed by an artificial intelligence program trained through various types of pre-captured eye image data and actual shooting processes.

The training data may be images of various eye movements, and can be trained to distinguish between states suitable and unsuitable for phototherapy, such as eyes open, eyes closed, and abnormal eyeball movements.

As an example of the normality determination step, if the eye closure continues for a preset time or longer based on the extracted eye object, it can be determined that treatment is not possible and the phototherapy can be interrupted or terminated.

In addition, as an example of the normality determination step, if the eye closure continues for a preset time or longer based on the extracted eye object, it can be determined that treatment/care cannot proceed, and while interrupting the phototherapy, a stimulus such as sound or vibration can be applied to the user to attempt to change to a state where phototherapy/photocare is possible.

After the stimulus is applied to the user, the eye object detection step to the normality determination step can be re-performed, and if it is determined that the eyes are open, the phototherapy/photocare can be resumed.

Regarding the artificial intelligence program, at least one of the eye object detection step, the eye object extraction step, and the normality determination step may be performed by an artificial intelligence program.

In particular, at least one of the eye object detection step and the normality determination step may be performed by an artificial intelligence program.

With the above configuration, by acquiring image of the eyes of the user by the camera unit (300), it is possible to check the light irradiation state during light irradiation by the pair of light modules (100), thereby enabling more effective control of the light irradiation.

Meanwhile, the eye object detection step, the eye object extraction step, and the normality determination step, as described above, may be performed by an app on a smart device such as a smartphone, smart pad, or dedicated device connected via wireless communication such as Bluetooth, instead of being performed by the image processing unit installed in the main body part (12).

For this purpose, the smart device may be installed with an app for wireless control of the system for eye disease treatment according to the present invention, reception of eye images, etc., from the system for eye disease treatment.

Meanwhile, in performing the normality determination step, since the image of the extracted eye object includes a reflection image of the treatment light formed on the retina, it is possible to confirm whether the treatment light is normally irradiated to the user's eyes by comparing the positions of the pupil in the eye object and the reflection image of the treatment light.

If the comparison of the positions of the pupil in the eye object and the reflection image of the treatment light shows a discrepancy, the error can be calculated to instruct the correction of the horizontal position with respect to the user's eye.

The horizontal position can be defined as the position of the light module (100) in the direction approaching and moving away from the nose, centered on the nose, while worn on the user's face.

Meanwhile, as a result of conducting animal tests using the system for eye disease treatment having the above-described configuration, it was confirmed that there is an effect of treating and alleviating eye diseases such as dry eye syndrome, macular degeneration, and macular edema.

That is, the system for eye disease treatment according to the present invention can be used as an eye disease treatment device for the treatment of eye diseases such as dry eye syndrome, macular degeneration, and macular edema.

### Test Example 1 (Dry Eye Syndrome Test)

a. Test Conditions

### 1) Test Cells

in vitro test cell: HCE-T cell (Human corneal epithelial cell line)

### 2) Media and Reagents

### a) Cell Culture

DMEM, 10% FBS, 1% Antibiotics, Trypsin

### b) Analysis Reagents

MTT (3-(4,5-dimethylthiazol-2-yl)-2,5-diphenyl-2H-tetrazolium bromide)
DMSO

### 3) In Vivo Test Animals:

a) Test Animal Strain: C57BL/6
b) Test Animal Sex/Quantity: Female/15 mice
c) Test Animal Weight/Age: 18-20 g/7 weeks old
d) Dry Eye Induction
   Subcutaneous injection of saline solution containing 0.5 mg/0.2 ml of Scopolamine hydrobromide 4 times daily for 17 days

### 4) Test Staining Reagents

### a) H&E Stain Reagents

Hematoxylin, Eosin
EtOH, Xylene

### b) Immunohistochemistry (IHC) Reagents

Primary antibody:
: IL-6 (abcam)
: IL-1 (abcam)
: TNF-α (abcam)
Secondary antibody
: Horse anti-mouse IgG ab (Vector)
: Goat anti-Rabbit IgG ab (Vector)
ABC kit (Vector)
DAB stain (Vector)

### 5) Test Equipment

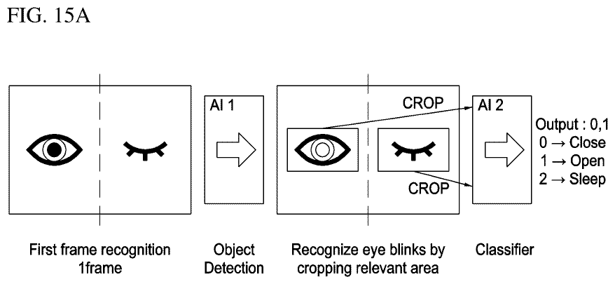

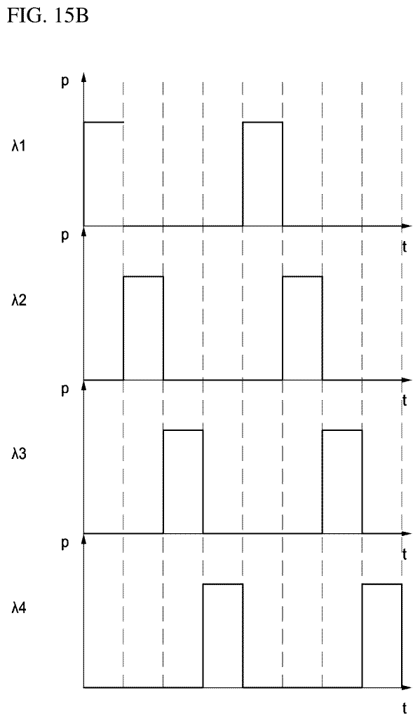

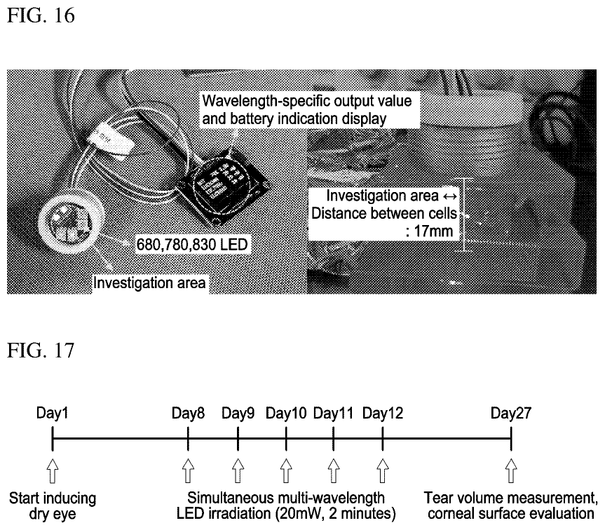

Considering that this test is for verifying the effectiveness of the system for eye disease treatment according to the present invention, the test equipment was configured as shown in FIG. 16.

### a) Wavelength Conditions

680 nm, 780 nm, 830 nm LEDs, where each wavelength can vary by ±10 nm.

### b) Light Irradiation Intensity (Irradiation Energy is the Average Light Output of Each LED)

12.3 mW (680 nm)+14.9 mW (780 nm)+10.6 mW (830 nm)=37.8 mW/cm², 2 minutes irradiation

Approximately 4.5 J/cm² b.

b. Test Method

### 1) In Vitro Safety Evaluation of Multi-Wavelength LED (Test Equipment Shown in FIG. 16) on Human Corneal Epithelial Cells

Culture HCE-T cells in media.
At 80% confluency, dispense into a 12-well plate.
Using a multi-wavelength (680 nm±10 nm, 780 nm±10 nm, 830 nm±10 nm) LED light source module, proceed with light irradiation on the plate where HCE-T cells are dispensed.
a) Irradiate light for different durations (0, 0.5, 1, 2, 4, 8 minutes) and check cell viability after 24 hours to confirm cell survival rate (MTT assay).
b) Fix light irradiation at 37.8 mW/cm², 2 minutes (4.5 J/cm²) and check cell proliferation rate through a proliferation assay for 0, 24, 48, 72 hours (MTT assay).

### \textcircled{1} MTT Analysis (MTT Assay)

Corneal epithelial cells (HCE-T) are cultured in an incubator under conditions of 5% CO₂, 37 °C using DMEM (Dulbecco's Modified Eagle Medium).
Corneal epithelial cells are seeded in a 12-well plate, and after 24 hours, irradiation with a multi-wavelength LED light source is performed.
The light source is irradiated by aligning the cylindrical end of the LED to one well of the 12-well plate where cells are seeded. (Distance from cylinder to cells: 17 mm)
After culturing for 0, 24, 48, 72 hours, the medium is removed, 50 μL of MTT solution and 100 μL of medium are added, and reacted for 4 hours in an incubator, protected from light.
After 4 hours, the reactants are removed, 100 μL of DMSO is added, and the absorbance is measured at 595 nm using a spectrophotometer.

### 2) In Vivo Efficacy Evaluation of Multi-Wavelength LED on Dry Eye Animal Model

### \textcircled{1} Dry Eye Induction

To induce a dry eye animal model, scopolamine hydrobromide was administered subcutaneously in the abdominal cavity at a concentration of 2.5 mg/mL, 4 times a day for 17 days.
One week after dry eye induction, light irradiation was performed 5 times on the entire eye area using a multi-wavelength LED light source module (wavelengths: 680 nm±10 nm, 780 nm±10 nm, 830 nm±10 nm).
After preparing 30 models, 5 animals per test group were randomly selected for the test.
After 17 days, tear volume and corneal surface irregularity were visually evaluated, and then tissues were extracted.
The test groups were divided into 3 groups as shown in Table 1 and proceeded according to the schedule in FIG. 17.

TABLE 1

| | |
|---|---|
| Control | Normal control group |
| (n = 5) | (saline solution) |
| Dry eye | Group with dry eye induced |
| (n = 5) | by Scopolamine hydrobromide |
| Dry eye + | Group with dry eye induction |
| Tx (n = 5) | followed by LED irradiation |

### \textcircled{2} Visual Observation

On the 17th day of dry eye induction, tear volume was measured for 1 minute using Absorbent Paper Point size 30.
After immobilizing the mouse, the extent of tear absorption by the paper between the eyelid and the eye was analyzed.
To evaluate corneal irregularity, intracorneal ring segments were imaged with a dissecting microscope, and the corneal surface irregularity observed in the control group (no treatment) was evaluated on a 4-point scale. The absence of irregularity was set to score 0, and the evaluation was conducted as shown in Table 2.

TABLE 2

| | | Corneal surface |
|---|---|---|
| Grade | | irregularity |
| score 0 | No damage | No damage |
| score 1 | slightly irregular | slightly irregular |
| score 2 | irregular less | irregular less |
| | than 50% circle | than 50% circle |
| score 3 | irregular greater | irregular greater |
| | than 50% circle | than 50% circle |

### \textcircled{3} Histological Observation

After the test, animals from each group were sacrificed, and to ensure objectivity, the right eye of all animals was uniformly extracted. After fixing in a 10% formalin solution, the tissue was infiltrated using 15% and 30% sucrose. It was hardened using OCT and cryosectioned at 10 μm.
H&E staining was performed to measure and analyze the corneal detachment density and area, and the thickness was measured and analyzed.
To confirm the degree of inflammation in the cornea, staining was performed for proinflammatory markers IL-6, IL-1β, and TNFα. The expression level was confirmed using DAB and analyzed with a tissue microscope.
   c. Test and Results

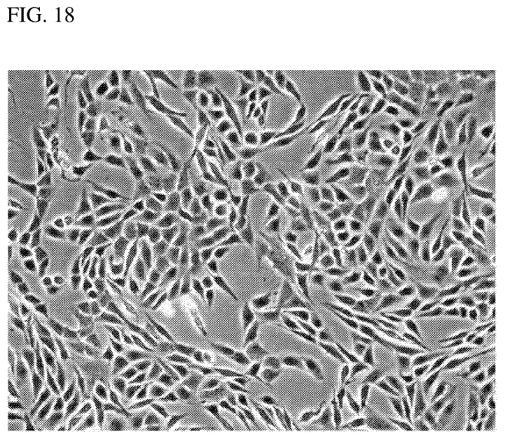

### 1) In Vitro Safety Evaluation of Multi-Wavelength LED on Human Corneal Epithelial Cells (see FIG. 18)

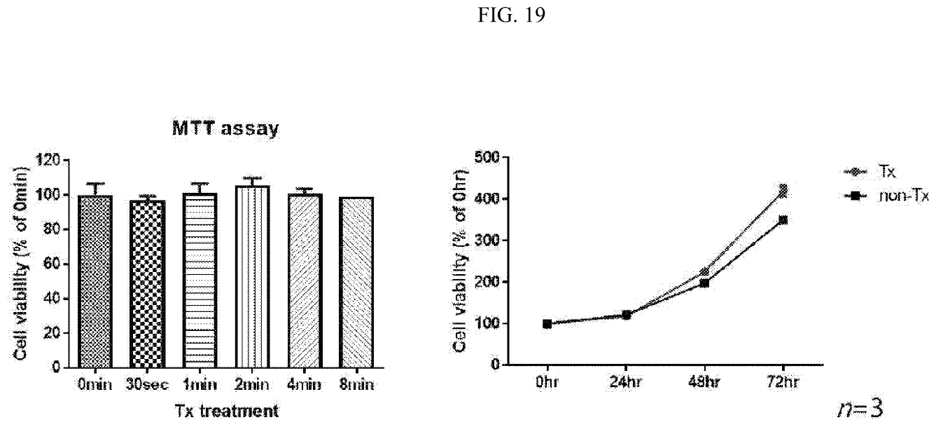

Measurement of cell survival rate through Cell viability 24 hours after light irradiation.
Multi-wavelength LED irradiation at various times (0 min, 0.5 min, 1 min, 2 min, 4 min, 8 min) did not reduce the cell survival rate of human corneal epithelial cells. That is, it is judged that there is no cytotoxicity, and the multi-wavelength LED shows a tendency to increase cell proliferation as shown in FIG. 19.

### 2) In Vivo Efficacy Evaluation of Multi-Wavelength LED on Dry Eye Animal Model (Table 3)

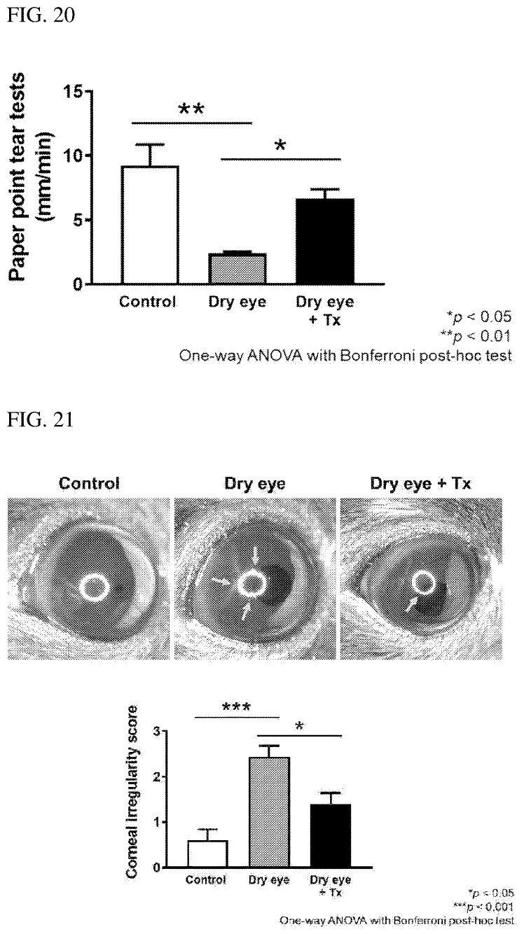

As a result of measuring the tear volume on the 17th day after inducing dry eye using absorbent paper point size 30, the normal control group was measured at an average of 9.2±1.4 mm, whereas the dry eye animal model showed a significant 73.8% decrease with an average of 2.4±0.1 mm.
On the other hand, the tear volume of the multi-wavelength LED treatment group was measured at 6.7±0.7 mm, showing a significant increase of 175.5% compared to the tear volume of the dry eye animal model. There is no significant difference between the control group and the LED treatment group.
Therefore, as shown in Table 3 and FIG. 20, the tear volume of the dry eye animal model used in this test was significantly reduced compared to the normal control group, and it is judged that the multi-wavelength LED significantly increases the tear volume reduced by dry eye.
Table 3 shows the change in tear volume due to dry eye and multi-wavelength LED treatment.

TABLE 3

| Tear | | | |
|---|---|---|---|
| measurement | Control | Dry eye | Dry eye + |
| (mm) | (n = 5) | (n = 5) | Tx (n = 5) |
| Average | 9.25 | 2.42 | 6.67 |
| SEM | 1.44 | 0.11 | 0.65 |

When dry eye occurs, the cornea is not protected by tears, leading to a roughening of the corneal surface. Therefore, in this test, after 17 days of dry eye induction, the irregularity of the corneal surface was observed with a stereomicroscope and blindly evaluated by three evaluators.
As a result of averaging the irregularity evaluation scores of the three evaluators, the normal control group was measured at an average of 0.6±0.2, as shown in Table 4 and FIG. 21, whereas the dry eye animal model showed a significant increase of 295.8% in the corneal surface irregularity score with an average of 2.4±0.3.
On the other hand, the corneal surface irregularity score of the multi-wavelength LED treatment group was measured at 1.4±0.2, showing a significant decrease of 41.1% compared to the irregularity score of the dry eye animal model. This value is similar to that of the normal control group. There is no significant difference between the control group and the LED treatment group.
Table 4 shows the evaluation of corneal surface irregularity due to dry eye and multi-wavelength LED treatment.

TABLE 4

| Corneal | | | |
|---|---|---|---|
| irregularity | Control | Dry eye | Dry eye + |
| score | (n = 5) | (n = 5) | Tx (n = 5) |
| Average | 0.60 | 2.38 | 1.40 |
| SEM | 0.22 | 0.25 | 0.22 |

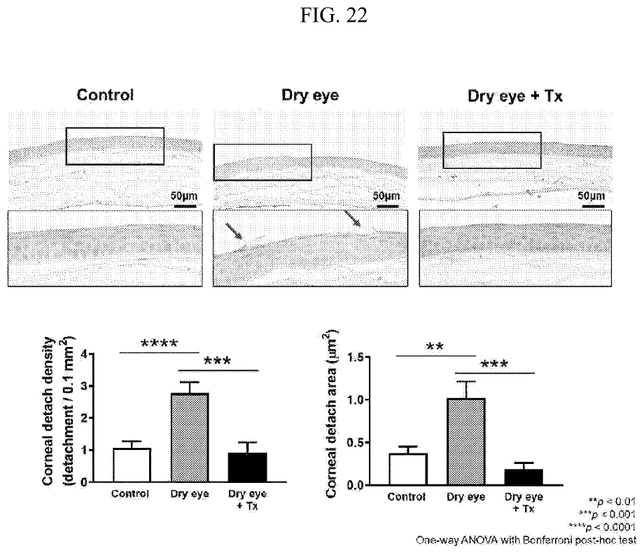

On the 17th day of dry eye induction, all animals were sacrificed, their eyeballs were enucleated, and H&E staining was performed on the cornea.
The corneal H&E staining images were analyzed using ImageJ software (NIH) to count the detached parts of the corneal surface, and the number was divided by a uniform area to measure and analyze the detached corneal surface density. The area of the detached cornea was also measured and analyzed with ImageJ.
As shown in FIG. 22, the density analysis result showed that the normal control group had an average density of 1.1±0.2, whereas the dry eye animal model had an average of 2.7±0.3, with the detached corneal surface density significantly increasing by 156.3%.
On the other hand, the detached corneal surface density of the multi-wavelength LED treatment group was measured at an average of 0.9±0.3, showing a significant decrease of 66.1% compared to the detached corneal surface density of the dry eye animal model.
The area analysis of the detached cornea showed that the normal control group had an average area of 0.4±0.1 μm², whereas the dry eye animal model had an average area of 1.0±0.2 μm², with the detached corneal area significantly increasing by 174%.
On the other hand, the detached corneal surface area of the multi-wavelength LED treatment group was measured at an average of 0.2±0.1 μm², showing a significant decrease of 81.4% compared to the detached corneal surface area of the dry eye animal model. There is no significant difference between the control group and the LED treatment group.
Table 5 and Table 6 respectively show the evaluation of detached corneal surface density and area due to dry eye and multi-wavelength LED treatment.

TABLE 5

| Corneal detach | Control | Dry eye | |
|---|---|---|---|
| density | (n = 5) | (n = 5) | Dry eye + Tx (n = 5) |
| Average | 1.06 | 2.72 | 0.92 |
| SEM | 0.22 | 0.33 | 0.31 |

TABLE 6

| Corneal detach area | Control | Dry eye | |
|---|---|---|---|
| (μm²) | (n = 5) | (n = 5) | Dry eye + Tx (n = 5) |
| Average | 0.37 | 1.02 | 0.19 |
| SEM | 0.08 | 0.19 | 0.07 |

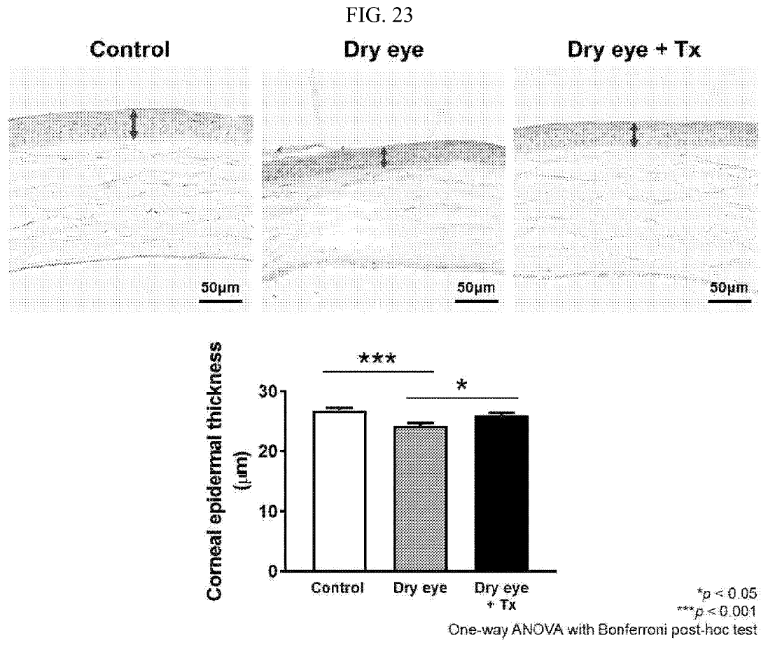

After measuring the detached corneal surface density and area with H&E staining, the thickness of the cornea was then measured and analyzed.
As shown in FIG. 23, the analysis of corneal thickness showed that the normal control group had an average thickness of 26.8±0.4 μm, whereas the dry eye animal model had an average of 24.2±0.5 μm, with the corneal thickness significantly decreasing by 9.6%.
On the other hand, the corneal thickness of the multi-wavelength LED treatment group was measured at an average of 25.9±0.5 μm, showing a significant increase of 7.1% compared to the corneal thickness of the dry eye animal model.
Table 7 shows the analysis results of corneal thickness changes due to dry eye and multi-wavelength LED treatment.

TABLE 7

| Corneal epidermal | Control | Dry eye | Dry eye + Tx |
|---|---|---|---|
| thickness (μm) | (n = 5) | (n = 5) | (n = 5) |
| Average | 26.77 | 24.20 | 25.91 |
| SEM | 0.41 | 0.54 | 0.45 |

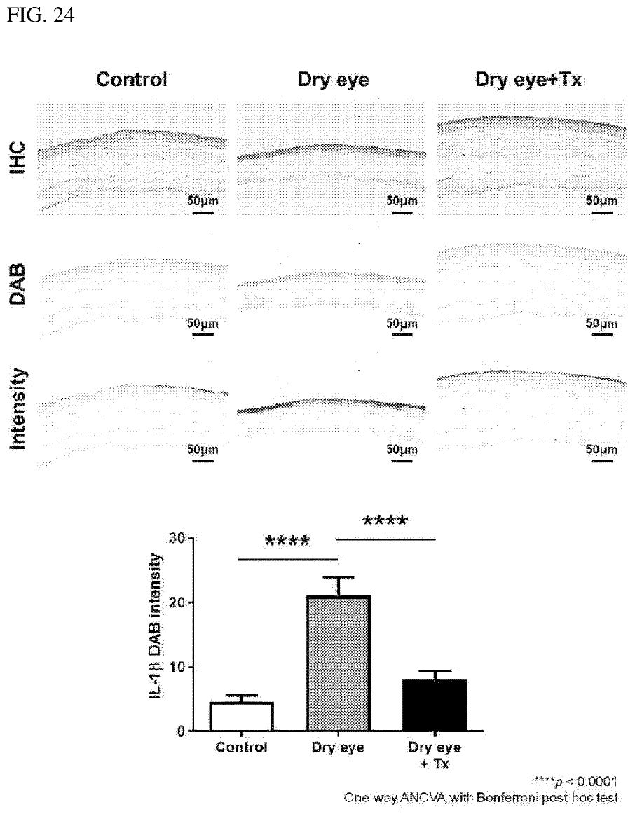

To measure the expression level of inflammatory cytokines, immunohistochemical staining was performed on sections of the enucleated eyeballs.
As shown in FIG. 24, after staining with IL-1β and analyzing the DAB image with the image analysis program ImageJ (NIH), the normal control group showed an average intensity of 4.6±1.0, whereas the dry eye animal model showed an average of 21.1±2.9, with the expression of IL-1β significantly increasing by 359.2%.
On the other hand, the IL-1β expression in the multi-wavelength LED treatment group was measured at an average of 8.1±1.2, showing a significant decrease of 61.5% compared to the IL-1β expression level of the dry eye animal model. There is no significant difference between the control group and the LED treatment group.
Table 8 shows the analysis results of IL-1β expression levels due to dry eye and multi-wavelength LED treatment.

TABLE 8

| | Control | Dry eye | |
|---|---|---|---|
| IL-1β DAB intensity | (n = 5) | (n = 5) | Dry eye + Tx (n = 5) |
| Average | 4.59 | 21.07 | 8.11 |
| SEM | 1.00 | 2.90 | 1.24 |

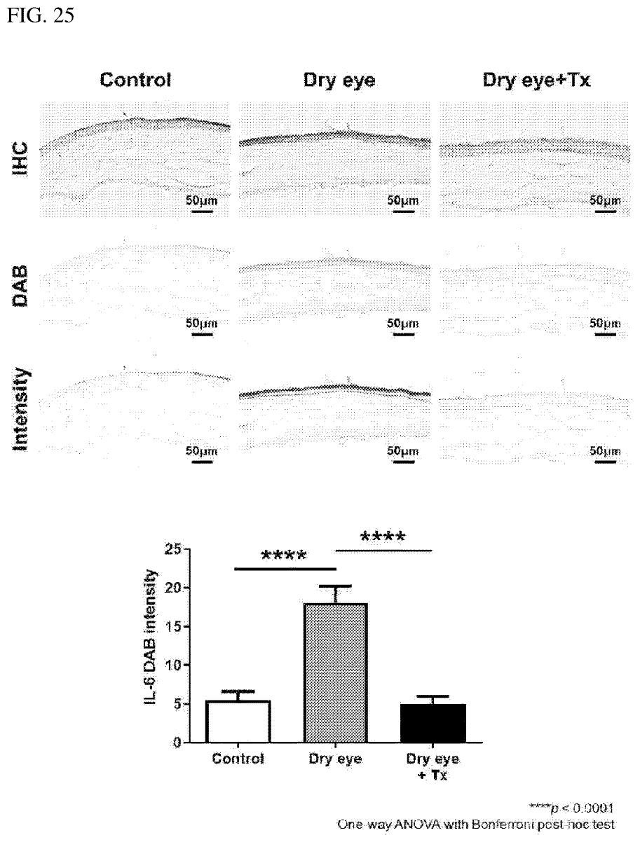

Next, to measure the expression level of IL-6 as an inflammatory cytokine, immunohistochemical staining was performed.
As shown in FIG. 25, after staining with IL-6 and analyzing the DAB image with the image analysis program ImageJ (NIH), the normal control group showed an average intensity of 5.4±1.1, whereas the dry eye animal model showed an average of 18.1±2.1, with the expression of IL-6 significantly increasing by 232.1%.
On the other hand, the IL-6 expression in the multi-wavelength LED treatment group was measured at an average of 5.1±0.9, showing a significant decrease of 72% compared to the IL-6 expression level of the dry eye animal model. There is no significant difference between the control group and the LED treatment group.
Table 9 shows the analysis results of IL-6 expression levels due to dry eye and multi-wavelength LED treatment.

TABLE 9

| | Control | Dry eye | Dry eye + |
|---|---|---|---|
| IL-6 DAB intensity | (n = 5) | (n = 5) | Tx (n = 5) |
| Average | 5.44 | 18.07 | 5.06 |
| SEM | 1.13 | 2.09 | 0.92 |

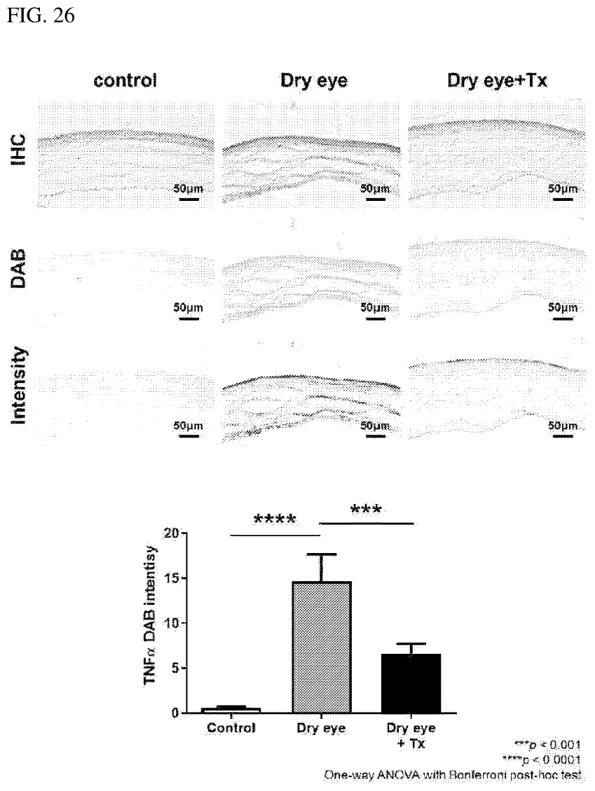

Next, to measure the expression level of TNFα as an inflammatory cytokine, immunohistochemical staining was performed.
As shown in FIG. 26, after staining with TNFα and analyzing the DAB image with the image analysis program ImageJ (NIH), the normal control group showed an average intensity of 0.6±0.2, whereas the dry eye animal model showed an average of 14.7±2.9, with the expression of TNFα significantly increasing by 2547%.
On the other hand, the TNFα expression in the multi-wavelength LED treatment group was measured at an average of 6.7±1.1, showing a significant decrease of 54.6% compared to the TNFα expression level of the dry eye animal model. There is no significant difference between the control group and the LED treatment group.
Table 10 shows the analysis results of TNFα expression levels due to dry eye and multi-wavelength LED treatment.

TABLE 10

| | Control | Dry eye | Dry eye + |
|---|---|---|---|
| TNFα DAB intensity | (n = 5) | (n = 5) | Tx (n = 5) |
| Average | 0.55 | 14.67 | 6.66 |
| SEM | 0.21 | 2.87 | 1.06 |

d. Test Result Conclusion
1) In this test, human corneal epithelial cells were used to evaluate the safety of a multi-wavelength LED that simultaneously irradiates three wavelengths of 630 nm, 780 nm, and 850 nm. As a result of treating human corneal epithelial cells with the multi-wavelength LED, it was confirmed that it did not reduce the cell survival rate depending on the energy or the cell's proliferation ability over time, thus confirming its non-harmfulness.
2) Subsequently, visual and histological observations were performed on a dry eye animal model, a mouse in which dry eye was induced for 17 days with scopolamine in a dry environment, and a Tx group irradiated 5 times with a multi-wavelength LED. First, as a result of measuring tear volume, the Tx group showed a significant increase in tear volume by 175.5% compared to the dry eye animal model.
3) Furthermore, in the corneal surface irregularity evaluation, a blind test by 3 evaluators confirmed that the corneal surface irregularity score, which had significantly increased by 295.8% in the dry eye animal model compared to the control group, was significantly reduced again by 41.1% in the Tx group compared to the expression level of the dry eye animal model.
4) Subsequently, in the histological evaluation, it was confirmed that the density of the detached corneal surface, which had increased by 156.3% in the dry eye animal model compared to the control group, was significantly reduced again by 66.1% in the Tx group. In addition, as a result of analyzing the area of the detached corneal surface, it was confirmed that the detached surface area, which had significantly increased by 174% in the dry eye animal model compared to the control group, was significantly reduced again by 81.4% in the Tx group compared to the expression level of the dry eye animal model.
5) As a result of measuring the corneal thickness, similarly to the previous results, it was confirmed that the corneal thickness, which had significantly decreased by 9.6% in the dry eye animal model compared to the normal control group, was significantly increased again by 7.1% in the Tx group compared to the expression level of the dry eye animal model.
6) In addition, DAB images of IL-1β, IL-6, and TNFα were analyzed through immunohistochemical staining performed to measure the expression levels of inflammatory cytokines. First, IL-1β, which was significantly increased by 359.2% in the dry eye animal model compared to the normal control group, was significantly decreased by 61.5% in the Tx group compared to the expression level of the dry eye animal model.
7) Subsequently, as a result of IL-6 analysis, it was confirmed that the IL-6 expression level, which was significantly increased by 232.1% in the dry eye animal model compared to the normal control group, was significantly reduced again by 72% in the Tx group.
8) The TNFα result also confirmed that the TNFα expression level, which was significantly increased by 2547% in the dry eye animal model compared to the normal control group, was significantly reduced by 54.6% in the Tx group compared to the expression level of the dry eye animal model.
9) Based on these test results, it is concluded that the multi-wavelength LED has no toxicity to corneal epithelial cells, and in an animal model with induced dry eye, it significantly reduces the levels of inflammatory cytokines induced in the cornea by dry eye, and significantly increases tear volume, thereby recovering corneal damage.

### Test Example 2 (Macular Degeneration Test)

a. Test Conditions

### 1) Test Cells

in vitro test cell: ARPE-19 cell (Human Retinal Pigment Epithelial Cell line, ATCC, #CRL-2302)

### 2) Media and Reagents

### a) Cell Culture

DMEM, 10% FBS, 1% Antibiotics, Trypsin

### b) Analysis Reagents

MTT (3-(4,5-dimethylthiazol-2-yl)-2,5-diphenyl-2H-tetrazolium bromide)
DMSO

### 3) In Vivo Test Animals:

a) Test Animal Strain: SD rat
b) Test Animal Sex/Quantity: Male/22 rats
c) Test Animal Weight/Age: 150-200 g/6 weeks old

### 4) Macular Degeneration Induction

Saline solution containing 35 mg/kg of NaIO₃

### 4) Test Staining Reagents

a) H&E stain reagents
Hematoxylin, Eosin
EtOH, Xylene
   b) TUNEL stain reagents
FITC TUNEL assay kit (BioActs)
PI staining (sigma)

### c) Immunohistochemistry (IHC) Reagents

Primary antibody
: RPE65 (abcam)
: Rhodopsin (abcam)
: PKC-α (Santacruz)
Secondary antibody
: Horse anti-Rabbit IgG ab, Alexa Fluor™ 555 (ThermoFisher)
: Goat anti-Mouse IgG ab, Alexa Fluor™ 488 (ThermoFisher)
DAPI (4′,6-diamidino-2-phenylindole)

### 5) Test Equipment

Considering that this test is for verifying the effectiveness of the system for eye disease treatment according to the present invention, the test equipment was configured as shown in FIG. 16.

### a) Wavelength Conditions

680 nm, 780 nm, 830 nm LEDs, where each wavelength can vary by ±10 nm.

### b) Light Irradiation Intensity (Irradiation Energy is the Average Light Output of Each LED)

12.3 mW (680 nm)+14.9 mW (780 nm)+10.6 mW (830 nm)=37.8 mW/cm², 2 minutes irradiation
Approximately 4.5 J/cm²
   b. Test Method

### 1) In Vitro Safety Evaluation of Multi-Wavelength LED on Retinal Pigment Epithelial Cells

Culture retinal pigment epithelial cells, ARPE-19 cells, in media.
At 80% confluency, dispense into a 12-well plate.
Using a multi-wavelength (680 nm±10 nm, 780 nm±10 nm, 830 nm±10 nm) LED light source module, proceed with light irradiation on the plate where ARPE-19 cells are dispensed.
a) Irradiate light for different durations (0, 0.5, 1, 2, 4, 8 minutes) and check cell viability after 24 hours to confirm cell survival rate (MTT assay).
b) Fix light irradiation at 37.8 mW/cm², 2 minutes (4.5 J/cm²) and check cell proliferation rate through a proliferation assay for 0, 24, 48, 72 hours (MTT assay).

### 1 MTT Analysis (MTT Assay)

Retinal epithelial cells (ARPE-19) are cultured in an incubator under conditions of 5% CO₂, 37 °C using DMEM (Dulbecco's Modified Eagle Medium).
Retinal epithelial cells are seeded in a 12-well plate, and after 24 hours, irradiation with a multi-wavelength LED light source is performed.
The light source is irradiated by aligning the cylindrical end of the LED to one well of the 12-well plate where cells are seeded (Distance from cylinder to cells: 17 mm).
After culturing for 0, 24, 48, 72 hours, the medium is removed, 50 μL of MTT solution and 100 μL of medium are added, and reacted for 4 hours in an incubator, protected from light.
After 4 hours, the reactants are removed, 100 μL of DMSO is added, and the absorbance is measured at 595 nm using a spectrophotometer.

### 2) In Vivo Efficacy Evaluation of Multi-Wavelength LED on NaIO₃-Treated Macular Degeneration Animal Model

The test was conducted with a total of 3 groups as shown in Table 11.
A minimum of 6 SD rats per group were secured for all groups.
Inject 35 mg/kg of NaIO₃ into the tail vein of 6-week-old SD rats.
Extract tissues at 1, 2, 3, 4, and 7 days after NaIO₃ injection to check the degree of ONL reduction in the retina.
After injection, irradiate the rat's eyes with 37.8 mW/cm², for 2 minutes (4.5 J/cm²) (5 times).
On the 7th day after NaIO₃ injection, extract tissues for subsequent experiments.

TABLE 11

| | |
|---|---|
| Control (n = 6) | Normal control group (saline solution) |
| Dry AMD 7 day (n = 8) | Group on the 7th day after 35 mg/kg |
| | NaIO₃ injection |
| Dry AMD + LED | Group on the 7th day after 35 mg/kg |
| 7 day (n=) | NaIO₃ injection + 5 light irradiations |

### \textcircled{1} Histological Observation (See Table 12)

Animals from each group were sacrificed at each period, eye tissues were extracted, fixed in 10% formalin solution, and then infiltrated with 15% and 30% sucrose. The tissue was hardened using OCT and cryosectioned at 10 μm.
H&E staining was confirmed with a tissue microscope, and the ONL of the retina was measured and analyzed.
TUNEL staining was confirmed with a confocal microscope, and the number of TUNEL-positive cells in the ONL of the retina was counted and analyzed.
PKC-α was confirmed with a confocal microscope, and the number of PKC-α positive cells in the OPL (Outer plexiform layer) and INL (Inner nuclear layer) was counted and analyzed.
Rhodopsin was confirmed with a confocal microscope, and the fluorescence expression intensity of Rhodopsin in the PR (photoreceptor layer) was analyzed using the image analysis program ImageJ (NIH).

TABLE 12

| | |
|---|---|
| Hematoxylin & | Confirm thickness of retinal ONL (Outer |
| Eosin (H&E) stain | nuclear layer) |
| TUNEL stain | Confirm cell apoptosis in retinal ONL |
| PKC-α stain | Confirm Bipolar cells |
| Rhodopsin, RPE 65 | Confirm Rod photoreceptors and RPE (Retinal |
| | pigment epithelium) |

c. Test and Results

### 1) In Vitro Safety Evaluation of Multi-Wavelength LED on Retinal Pigment Epithelial Cells

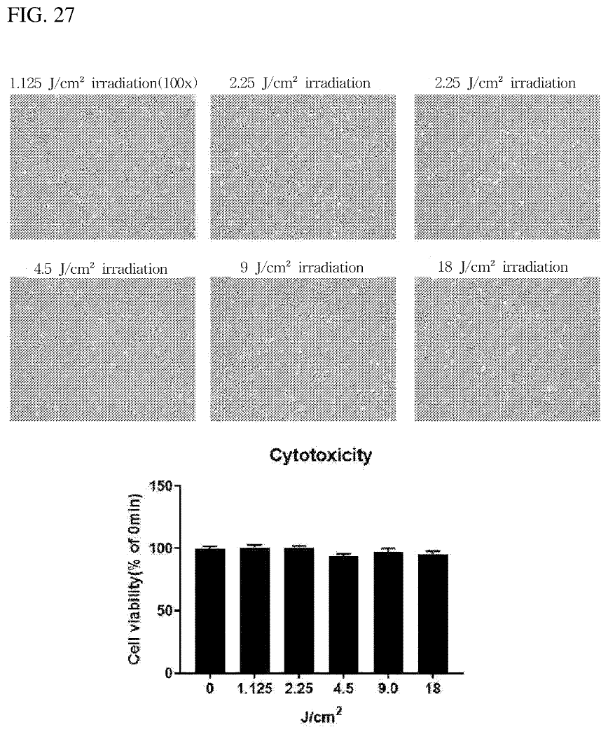

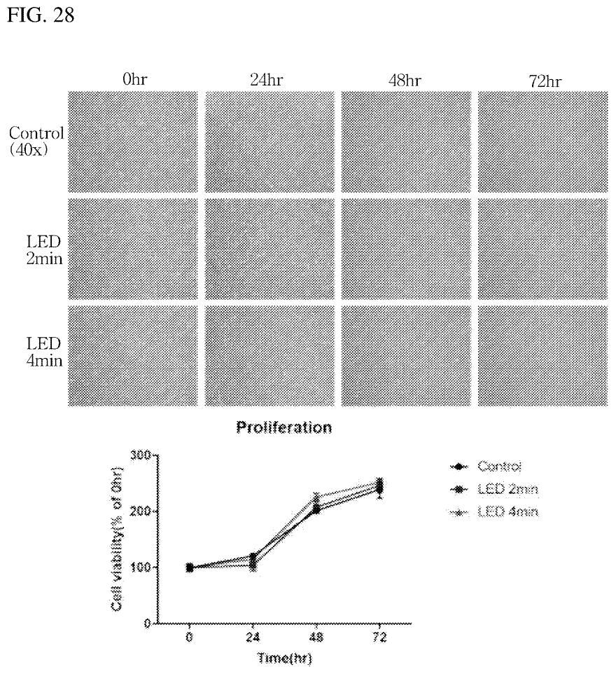

Measurement of cell survival rate through Cell viability 24 hours after light irradiation.
As shown in FIG. 27, multi-wavelength LED irradiation at various times (0 min, 0.5 min, 1 min, 2 min, 4 min, 8 min) did not reduce the cell survival rate of retinal pigment epithelial cells. Therefore, it is judged that there is no cytotoxicity due to multi-wavelength LED irradiation.
Measurement of cell proliferation rate through Proliferation assay for 0, 24, 48, 72 hours after light irradiation.
It was confirmed that multi-wavelength LED irradiation does not cause problems in the cell proliferation of retinal pigment epithelial cells over time, as shown in FIG. 28.

### 2) In Vivo Efficacy Evaluation of Multi-Wavelength LED on NaIO₃-Treated Macular Degeneration Animal Model

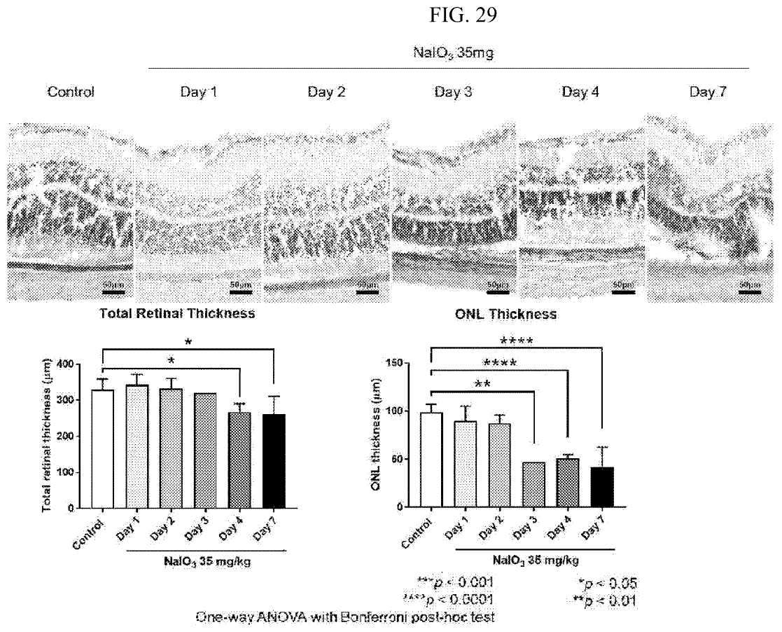

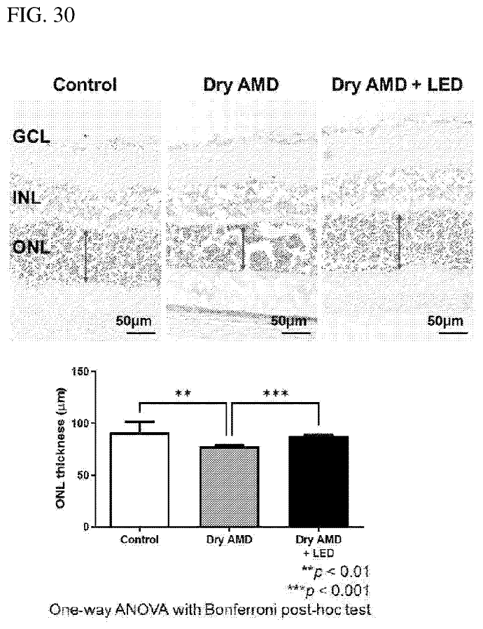

After 1, 2, 3, 4, and 7 days of NaIO₃ treatment, eyeballs were enucleated and H&E staining was performed.
As shown in FIG. 29, by analyzing the total retinal thickness and ONL thickness, the condition for the macular degeneration animal model was established as 7 days after NaIO₃ treatment.
After 7 days of NaIO₃ and multi-wavelength LED treatment, eyeballs were enucleated and H&E staining was performed.
The ONL (outer nuclear layer), composed of the nuclei of photoreceptor cells, has a layered structure of photoreceptor nuclei. Therefore, by measuring the thickness of the ONL, a decrease in the number of ONL photoreceptor cells due to macular degeneration was confirmed.
As shown in FIG. 30, analysis of the ONL thickness in the retina showed a significant decrease of 14.7% in the macular degeneration animal model compared to the normal control group, and a significant increase of 12.3% in the multi-wavelength LED treatment group compared to the macular degeneration animal model.

Table 13 shows the change in retinal ONL thickness due to macular degeneration and multi-wavelength LED treatment.

TABLE 13

| | Control | Dry AMD | Dry AMD + LED |
|---|---|---|---|
| ONL thickness (μm) | (n = 6) | (n = 8) | (n = 8) |
| Average | 91.25 | 77.83 | 87.38 |
| SEM | 9.93 | 0.99 | 1.46 |

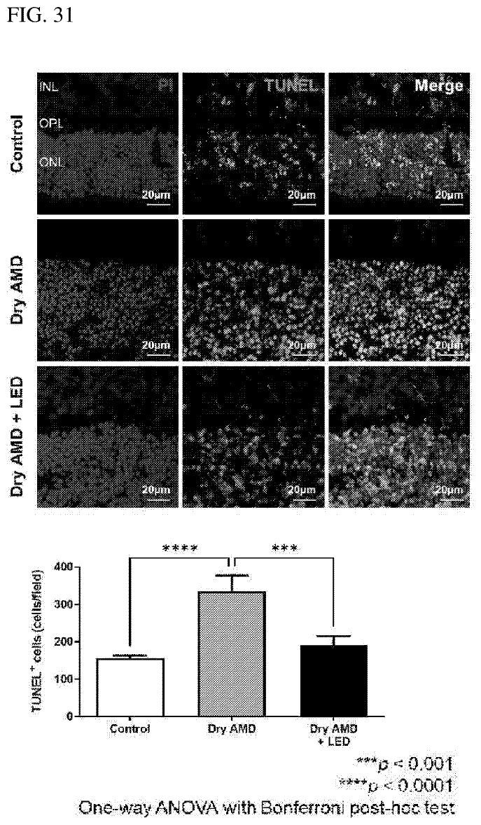

After 7 days of NaIO₃ and multi-wavelength LED treatment, the enucleated eyes were sectioned and a TUNEL assay was performed to check for apoptosis in the tissue.
As shown in FIG. 31, the TUNEL reagent is a method for fluorescently staining cells undergoing apoptosis. As a result of counting TUNEL-positive apoptotic cells in the retinal ONL, there was a significant increase of 115.1% in the macular degeneration animal model compared to the normal control group, and a significant decrease of 42.8% in the multi-wavelength LED treatment group compared to the macular degeneration animal model. There is no significant difference between the control group and the LED treatment group.
Table 14 shows the counting results of TUNEL-positive cells in the retina due to macular degeneration and multi-wavelength LED treatment.

TABLE 14

| TUNEL-positive | Control | Dry AMD | Dry AMD + LED |
|---|---|---|---|
| cells (count) | (n = 6) | (n = 8) | (n = 8) |
| Average | 155.78 | 335.12 | 191.52 |
| SEM | 7.23 | 40.88 | 23.06 |

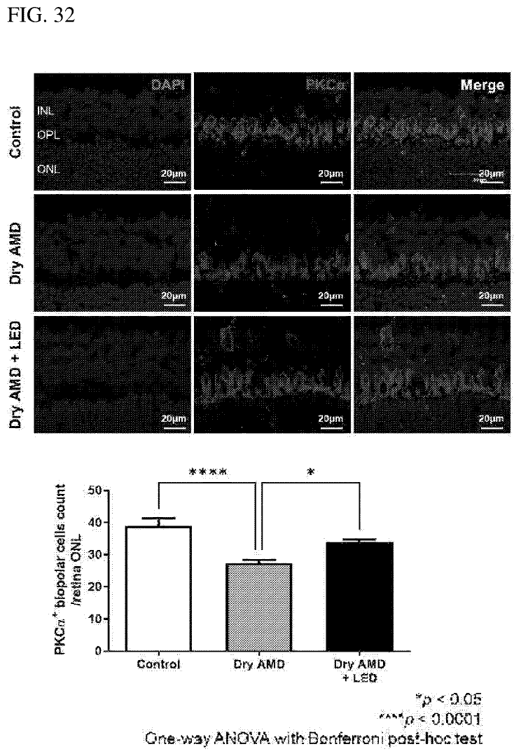

After 7 days of NaIO₃ and multi-wavelength LED treatment, the enucleated eyes were sectioned and immunofluorescence staining was performed to confirm the expression of PKCα, a bipolar cell marker in the retina.
As shown in FIG. 32, bipolar cells are mainly located in the inner nuclear layer (INL) of the retina, and they form synapses in both the OPL (outer plexiform layer) and IPL (inner plexiform layer), the layers above and below the INL. In the visual pathway, bipolar cells play a crucial role in transmitting electrochemical signals from photoreceptor cells to ganglion cells, so these bipolar cells were examined.
After PKCα immunofluorescence staining, counting PKCα-positive cells in the retina revealed a significant decrease of 18.4% in the macular degeneration animal model compared to the normal control group, and a significant increase of 7% in the multi-wavelength LED treatment group compared to the macular degeneration animal model. There is no significant difference between the control group and the LED treatment group.
Table 15 shows the counting results of PKCα-positive cells in the retina due to macular degeneration and multi-wavelength LED treatment.

TABLE 15

| PKCα-positive cells | Control | Dry AMD | Dry AMD + LED |
|---|---|---|---|
| (count) | (n = 6) | (n = 8) | (n = 8) |
| Average | 38.94 | 31.79 | 34.00 |
| SEM | 2.29 | 1.30 | 0.82 |

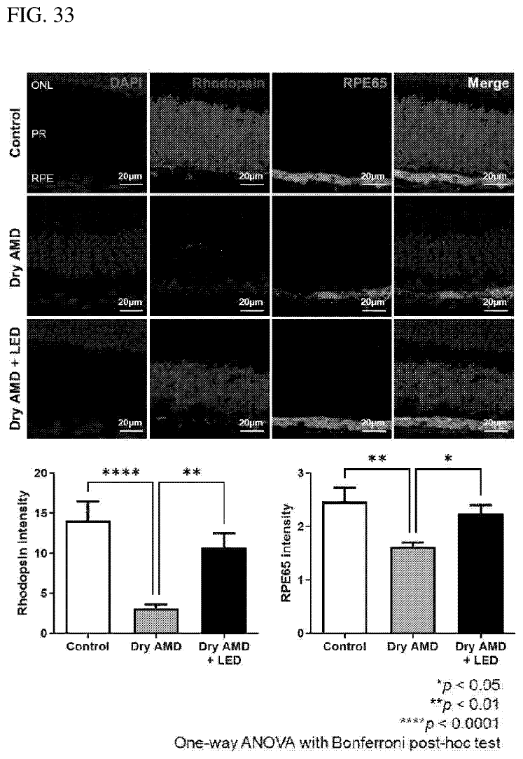

After 7 days of NaIO₃ and multi-wavelength LED treatment, the enucleated eyes were sectioned and immunofluorescence staining was performed to confirm the expression of Rhodopsin, a marker for Rods in the retinal PR, and RPE65, a marker for RPE.
Through Rhodopsin, a marker for Rods, the change in the amount of Rods, which are receptors in the retina responsible for vision that convert incoming light into electrical signals decipherable by the brain's visual processing area, was confirmed. Also, by measuring the fluorescence expression intensity of the RPE65 marker, the change in the RPE layer, which absorbs light entering the eye, due to macular degeneration and multi-wavelength LED was confirmed.
As shown in FIG. 33, after immunofluorescence staining, analysis of the Rhodopsin fluorescence expression intensity in the retinal PR using the ImageJ software (NIH) showed a significant decrease of 78.1% in the macular degeneration animal model compared to the normal control group, and a significant increase of 248.2% in the multi-wavelength LED treatment group compared to the macular degeneration animal model. There is no significant difference between the control group and the LED treatment group.
After immunofluorescence staining, analysis of the fluorescence expression intensity of RPE65, a marker for the retinal RPE layer, using the ImageJ software (NIH) showed a significant decrease of 34.2% in the macular degeneration animal model compared to the normal control group, and a significant increase of 38.3% in the multi-wavelength LED treatment group compared to the macular degeneration animal model. There is no significant difference between the control group and the LED treatment group.
Table 16 shows the analysis results of Rhodopsin fluorescence expression intensity in the PR due to macular degeneration and multi-wavelength LED treatment, and Table 17 shows the analysis results of RPE65 fluorescence expression intensity due to macular degeneration and multi-wavelength LED treatment.

TABLE 16

| Rhodopsin fluorecent | Control | Dry AMD | Dry AMD + LED |
|---|---|---|---|
| intensity | (n = 6) | (n = 8) | (n = 8) |
| Average | 14.09 | 3.09 | 10.76 |
| SEM | 2.27 | 0.47 | 1.65 |

TABLE 17

| RPE65 fluorecent | Control | Dry AMD | Dry AMD + LED |
|---|---|---|---|
| intensity | (n = 6) | (n = 8) | (n = 8) |
| Average | 2.47 | 1.62 | 2.25 |
| SEM | 0.25 | 0.07 | 0.15 |

d. Test Result Conclusion
1) In this study, retinal pigment epithelial cells were used to evaluate the safety of a multi-wavelength LED that simultaneously irradiates three wavelengths of 680 nm, 780 nm, and 830 nm. It was confirmed that treating retinal pigment epithelial cells with the multi-wavelength LED did not reduce cell survival rate based on energy or cell proliferation ability over time.
2) Subsequently, to establish a macular degeneration animal model, mice were treated with 35 mg/kg of sodium iodide, and after analyzing the total retinal thickness and ONL thickness, it was confirmed that the damage was most significant on the 7th day post-injection, thus establishing the dry macular degeneration animal model.
3) When the macular degeneration animal model was treated with the multi-wavelength LED, the retinal ONL thickness significantly increased by 12.3% compared to the macular degeneration model.
4) The TUNEL assay, which can identify apoptotic cells, showed that the multi-wavelength LED significantly reduced the number of apoptotic cells by 42.8% compared to the macular degeneration animal model.
5) Analysis of the number of cells expressing PKCα, a marker for retinal bipolar cells, showed that the multi-wavelength LED significantly increased the number of PKCα-positive cells by 7% compared to the macular degeneration animal model.
6) Analysis of the Rhodopsin fluorescence expression intensity, a marker for Rods in the retinal PR, showed that the multi-wavelength LED significantly increased the Rhodopsin fluorescence expression intensity by 248.2% compared to the macular degeneration animal model.
7) Analysis of the fluorescence expression intensity of RPE65, a marker for the retinal RPE layer, showed that the multi-wavelength LED significantly increased the RPE65 fluorescence expression intensity by 38.3% compared to the macular degeneration animal model.
8) Based on these test results, it is concluded that the multi-wavelength LED has no toxicity to retinal pigment epithelial cells and significantly recovers the reduction of bipolar cells, which play a crucial role in the visual pathway, and the damage to the Rods and RPE layer, which are receptors for visual information, in the macular degeneration animal model, thereby significantly recovering the reduction in retinal ONL thickness and cell apoptosis.
9) Based on this test result, it is judged that when irradiating the user's eyes with the wavelength and light irradiation intensity conditions of the previously described test equipment for the treatment or alleviation of macular degeneration/macular edema, it not only does not damage the user's cornea as in Test Example 1, but can also improve the condition of the user's cornea.

### Test Example 3 (Macular Edema Test)

a. Test Conditions
1) in vivo Test Animals:
a) Test Animal Strain: SD rat
b) Test Animal Sex/Quantity: Male/18 rats
c) Test Animal Weight/Age: 150-200 g/6 weeks old

### 3) Macular Edema Induction

Saline solution containing 55 mg/kg of Streptozotocin (STZ)

### 3) Test Staining Reagents

### a) H&E Stain Reagents

Hematoxylin, Eosin
EtOH, Xylene

### 4) Test Equipment

Considering that this test is for verifying the effectiveness of the system for eye disease treatment according to the present invention, the test equipment was configured as shown in FIG. 16.

### a) Wavelength Conditions

680 nm, 780 nm, 830 nm LEDs, where each wavelength can vary by ±10 nm.

### b) Light Irradiation Intensity (Irradiation Energy is the Average Light Output of Each LED)

12.3 mW (680 nm)+14.9 mW (780 nm)+10.6 mW (830 nm)=37.8 mW/cm², 2 minutes irradiation
Approximately 4.5 J/cm²
   b. Test Method
An animal model of diabetic retinopathy (macular edema; ME) can be established through the induction of diabetes.
To induce a diabetic retinopathy (macular edema) animal model, STZ was administered subcutaneously in the abdominal cavity once at a concentration of 55 mg/kg.
From the day after administration, light irradiation was performed 3 times on the entire eye area using a multi-wavelength (680 nm, 780 nm, 830 nm) LED light source module.
The test was conducted with a total of 3 groups as shown in the table below (see Table 18).
The animals were raised for 30 days from the date of administration, and at the end of the experiment, animals from each group were sacrificed and their eye tissues were extracted. The tissues were fixed in 10% formalin solution and then infiltrated with 15% and 30% sucrose. They were hardened using OCT and cryosectioned at 10 μm.
H&E staining was performed to measure and analyze the retinal ONL (Outer nuclear layer).

TABLE 18

| | |
|---|---|
| Control (n = 2) | Normal control group (saline solution) |
| STZ (n = 8) | Group with macular edema induced by Streptozotocin |
| STZ + LED | Group with macular edema induction followed by |
| (n = 8) | LED irradiation |

c. Test and Results

### 1) Production and Verification of Diabetic Retinopathy (Macular Edema) Animal Model

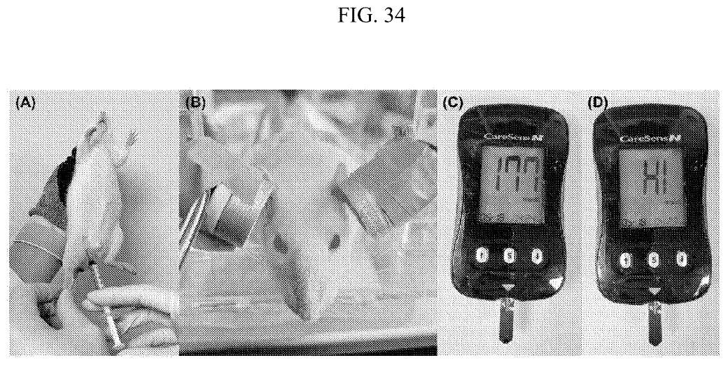

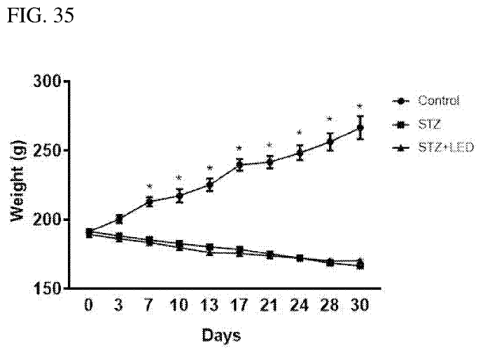

To produce a diabetic retinopathy (macular edema) animal model, STZ, which induces diabetes, was administered intraperitoneally once at 55 mg/kg.
From the day after STZ administration, LED irradiation was performed 3 times a week (2 minutes per session, 4.5 J/cm²).
As shown in FIG. 34, one week after STZ administration, blood glucose was measured. Only animals with blood glucose levels of 600 mg/dL or higher (indicated as High on the blood glucose meter) compared to normal (100-200 mg/dL) were selected and divided into diabetic retinopathy (macular edema) and LED irradiation groups.
Subsequently, they were raised for 30 days, and body weight changes were observed at 3-4 day intervals.
As shown in FIG. 35, a significant change in body weight was observed between the normal control group and the STZ, STZ+LED groups from day 7 onwards.

### 2) In Vivo Efficacy Evaluation of Multi-Wavelength LED on Diabetic Retinopathy (Macular Edema) Animal Model

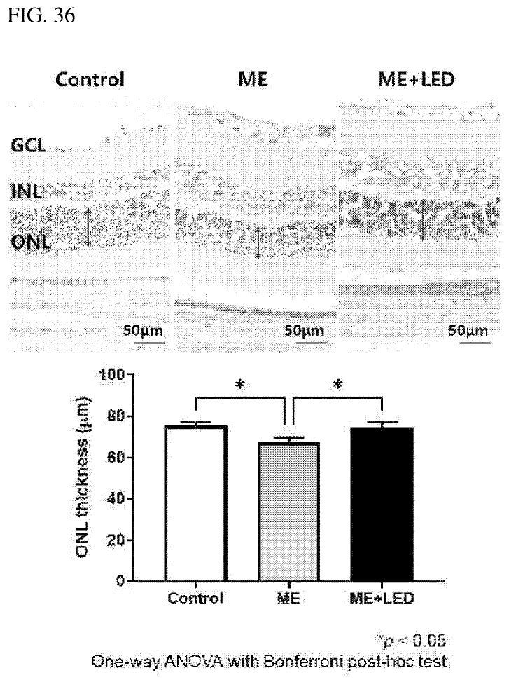

To produce a diabetic retinopathy (macular edema) animal model, STZ, which induces diabetes, was administered, and after raising for 30 days, the animals were sacrificed, and H&E staining was performed on the retina of the enucleated eyes.
As shown in FIG. 36, the ONL (outer nuclear layer), composed of the nuclei of photoreceptor cells, has a structure with multiple layers of photoreceptor nuclei. In cases of retinal damage, its thickness decreases due to the reduction of photoreceptor cells. Therefore, in this test, the thickness of the ONL was measured to confirm the decrease in the number of ONL photoreceptor cells due to diabetic retinopathy (macular edema).
The retinal H&E staining images were analyzed to measure the ONL thickness and analyze the changes caused by the multi-wavelength LED.
The ONL thickness analysis revealed that the normal control group had an average thickness of 75.4±1.7 μm, whereas the macular edema animal model had an average of 67.3±2.1 μm, showing a significant decrease in ONL thickness by 10.7%.
However, the ONL thickness of the multi-wavelength LED treatment group was measured at an average of 74.7±2.4 μm, showing a significant increase of 11% compared to the ONL thickness of the diabetic retinopathy (macular edema) animal model.

Table 19 shows the ONL thickness evaluation in the diabetic retinopathy (macular edema) animal model and after multi-wavelength LED treatment.

TABLE 19

| Corneal detach | | ME | ME + |
|---|---|---|---|
| density | Control | (macular edema) | LED |
| Average | 75.45 | 67.34 | 74.72 |
| SEM | 1.65 | 2.13 | 2.37 |

d. Test Result Conclusion
1) In this study, H&E staining was performed as a histological evaluation for a multi-wavelength LED that simultaneously irradiates three wavelengths of 680 nm, 780 nm, and 830 nm. When treated on a diabetic retinopathy (macular edema) animal model, the change in retinal ONL thickness was confirmed. It was confirmed that the ONL thickness was significantly reduced by 10.7% in the diabetic retinopathy (macular edema) animal model compared to the normal control group. There is no significant difference between the control group and the LED treatment group.
2) On the other hand, in the group irradiated with the multi-wavelength LED, it was confirmed that the ONL thickness was significantly increased again by 11% compared to the diabetic retinopathy (macular edema) animal model.
3) Based on these test results, it is concluded that the multi-wavelength LED significantly increases the reduced retinal ONL thickness in the diabetic retinopathy (macular edema) animal model, thereby restoring the reduced vision.

Meanwhile, the wavelengths and irradiation intensities of the lights irradiated by the test apparatus shown in FIG. 16 are as shown in Table 20.

TABLE 20

| Category | EDC680D | | EDC780D | | EDC830N | | Total |
|---|---|---|---|---|---|---|---|
| Sample | Wavelength | Intensity | Wavelength | Intensity | Wavelength | Intensity | Intensity |
| N# | (nm) | (mW/cm2) | (nm) | (mW/cm2) | (nm) | (mW/cm2) | (mW/cm2) |
| 1 | 678 | 12.618 | 772 | 14.685 | 822 | 10.281 | 37.584 |
| 2 | 677 | 11.708 | 772 | 14.579 | 823 | 10.498 | 36.785 |
| 3 | 677 | 12.558 | 772 | 14.616 | 822 | 10.823 | 37.997 |
| 4 | 677 | 12.568 | 772 | 15.072 | 822 | 10.461 | 38.101 |
| 5 | 678 | 12.164 | 772 | 15.236 | 823 | 10.747 | 38.147 |
| 6 | 677 | 12.246 | 773 | 15.092 | 822 | 10.814 | 38.152 |
| Average | — | 12.3103 | — | 14.88 | — | 10.604 | 37.7943 |

And through the above test results, the present invention can be summarized as follows.

First, the system for eye disease treatment according to the present invention is characterized in that it includes the optical care device described with reference to FIGS. 1 to 14, as well as a light module (100) that generates three or more lights with different wavelengths to irradiate the user's eyes, and the three or more lights include lights of a second wavelength of 680 nm±10 nm, a third wavelength of 780 nm±10 nm, and a fourth wavelength of 830 nm±10 nm.

Here, it is preferable that the sum of the irradiation intensity of the second to fourth wavelength lights is 35-40 mW/cm², and more preferably, the sum of the irradiation intensity of the second to fourth wavelength lights is 37.8 mW/cm².

In addition, it is preferable that the average value of the light irradiation intensity of the second wavelength light is 12.3 mW/cm², the average value of the light irradiation intensity of the third wavelength light is 14.9 mW/cm², and the average value of the light irradiation intensity of the fourth wavelength light is 10.6 mW/cm².

In addition, the energy of the second to fourth wavelength lights irradiated for 2 minutes may be irradiated to be less than 5.0 J/cm².

And the second to fourth wavelength lights may be simultaneously irradiated to the user's eyes.

The three or more lights may further include a light of a first wavelength of 590±10 nm.

The system for eye disease treatment according to the present invention having the above configuration can be used for the treatment or symptom relief of at least one eye disease among dry eye syndrome, macular edema, and macular degeneration.

Meanwhile, when the system for eye disease treatment according to the present invention is used for care/treatment/symptom relief of dry eye syndrome, considering that the irradiated light is irradiated to the cornea, it is preferable that the distance between the objective lens of the optical system constituting the light module (100) and the cornea is 1.5 cm to 2.0 cm, and more preferably 1.7 cm.

For example, when the system for eye disease treatment has a structure similar to glasses as shown in FIGS. 1 to 14, it is preferable that the distance between the objective lens of the light module (100) and the cornea is 1.5 cm to 2.0 cm, and more preferably 1.7 cm, in a worn state.

In addition, when the system for eye disease treatment according to the present invention is used for care/treatment/symptom relief of macular degeneration and/or macular edema, considering that the irradiated light is irradiated to the retina.

It is preferable that the distance is 3.9 cm to 4.4 cm, and more preferably 4.1 cm. For example, when the system for eye disease treatment has a structure similar to glasses as shown in FIGS. 1 to 14, it is preferable that the distance between the objective lens of the light module (100) and the cornea is 3.9 cm to 4.4 cm, and more preferably 4.1 cm, in a worn state.

For reference, the average distance between the cornea and the retina is 2.4 cm.

Meanwhile, although the treatment system for eye disease treatment, the eye disease treatment method, and the light module used therein according to the present invention have been described with an example of being used for the purpose of treating eye diseases, it is a matter of course that they can also be utilized as a system for eye disease treatment that maintains or enhances the active state of retinal cells by applying light stimulation to the retinal cells by irradiating the user's eyes with light.

That is, it is a matter of course that the treatment system for eye disease treatment according to the present invention can be utilized not only as a medical device for treating eye diseases but also as a health supplement device that can maintain or improve eye health.

When the present invention is used as a health supplement device for eye health rather than for treating eye diseases, it can be configured in various ways, such as being configured to irradiate a single light with a wavelength of 590±10 nm instead of being configured to irradiate a plurality of lights, of course.

Although the preferred embodiments of the present invention have been described above by way of example, the scope of the present invention is not limited to such specific embodiments, and may be appropriately modified within the scope described in the claims.

1. A system for eye disease treatment comprising: a main body module in which a light module for generating care light is provided, and which covers the eyes of a user so that the eyes of the user are irradiated with the light of the light module; and a wearing module which is coupled to the main body module, and which extends to a side surface of the head of the user so as to be worn by the user.

2. The system for eye disease treatment of claim 1, wherein the light module comprises:
an LED module including an LED substrate on which one or more LED devices are installed;
a barrel to one end of which the LED module is coupled;
one or more lenses installed in the barrel for diverging the light generated by the LED device; and
a diffusion member coupled to the barrel for diffusing light; and a fixing coupling part coupled to the barrel for fixing the lens and the diffusion member.

3. (canceled)

4. The system for eye disease treatment of claim 2,
wherein the LED device comprises: at least one of
a first LED device for irradiating light of a blue wavelength band;
a second LED device for irradiating light of a green wavelength band;
a third LED device for irradiating light of a red wavelength band;
a fourth LED device for irradiating light of an ultraviolet wavelength band; and
a fifth LED device for irradiating light of an infrared wavelength band.

5. (canceled)

6. (canceled)

7. (canceled)

8. The system for eye disease treatment of claim 1, wherein the system generates three or more lights with different wavelengths to irradiate a user's eyes,
and the three or more lights include lights of a second wavelength of 680 nm±10 nm, a third wavelength of 780 nm±10 nm, and a fourth wavelength of 830 nm±10 nm.

9. The system for eye disease treatment of claim 8,
wherein the sum of the irradiation intensity of the second to fourth wavelength lights is 35-40 mW/cm².

10. The system for eye disease treatment of claim 9,
wherein the sum of the irradiation intensity of the second to fourth wavelength lights is 37.8 mW/cm².

11. The system for eye disease treatment of claim 8,
wherein the average value of the light irradiation intensity of the second wavelength light is 12.3 mW/cm²,
the average value of the light irradiation intensity of the third wavelength light is 14.9 mW/cm², and
the average value of the light irradiation intensity of the fourth wavelength light is 10.6 mW/cm².

12. The system for eye disease treatment of claim 8,
wherein the second to fourth wavelength lights are simultaneously irradiated to the user's eyes.

13. The system for eye disease treatment of claim 8,
wherein the energy of the second to fourth wavelength lights irradiated for 2 minutes is irradiated to be less than 5.0 J/cm².

14. The system for eye disease treatment of claim 8,
wherein the three or more lights further include a light of a first wavelength of 590 nm±10 nm.

15. The system for eye disease treatment of claim 8,
wherein it is used for the treatment or symptom relief of at least one eye disease among dry eye syndrome, macular edema, and macular degeneration.

16.-25. (canceled)
